(19) United States
(12) Patent Application Publication
(10) Pub. No.: US 2022/0033846 A1
(43) Pub. Date: Feb. 3, 2022

(54) APP MODIFICATION VIA BASE EDITING USING THE CRISPR/CAS9 SYSTEM

(71) Applicants: Jacques P. TREMBLAY, Québec (CA); Joël ROUSSEAU, Québec (CA); Antoine GUYON, Québec (CA)

(72) Inventors: Jacques P. TREMBLAY, Québec (CA); Joël ROUSSEAU, Québec (CA); Antoine GUYON, Québec (CA)

(21) Appl. No.: 17/309,808

(22) PCT Filed: Dec. 20, 2019
(86) PCT No.: PCT/CA2019/051876

§ 371 Date: Jun. 18, 2021

Related U.S. Application Data
(60) Provisional application No. 62/783,573, filed on Dec. 21, 2018.

(51) Int. Cl. C12N 15/85; C12N 9/22; C12N 15/113

(52) U.S. Cl. CPC C12N 15/85; A61K 38/46; C12N 15/113; C12N 9/22

(57) ABSTRACT

Methods and products (e.g., guide RNAs or pegRNA and recombinant proteins) are described for decreasing amyloidogenic Aβ peptide levels produced by a cell, or the aggregation of the Aβ peptide, as well as uses of such methods and products, for example for the treatment of Alzheimer's disease and/or age-related cognitive decline in a subject in need thereof.

### CROSS REFERENCE TO RELATED APPLICATIONS

This application claims priority of U.S. provisional application Ser. No. 62/783,573 filed on Dec. 21, 2018, which is incorporated herein by reference in its entirety.

### SEQUENCE LISTING

This application contains a Sequence Listing in computer readable form entitled “17002_6_SeqList.txt”, created Dec. 20, 2019 and having a size of about 1,074 KB. The computer readable form is incorporated herein by reference.

### FIELD OF THE DISCLOSURE

The present disclosure relates to the introduction of modifications in the Amyloid 3 Precursor Protein (APP) gene via base or prime editing using the CRISPR/CAS9 system, as well as to uses thereof, for example to decrease the production of amyloidogenic Aβ peptides, and to prevent or treat Alzheimer's disease and/or age-related cognitive decline.

### BACKGROUND OF THE DISCLOSURE

More than 5% of the population of the Western world above the age of 60 is affected by dementia. Two thirds of these cases are due to Alzheimer's disease (AD)¹⁻³. The prevalence of AD doubles every 5 years after age 65. Thus, in persons older than 90 years of age there is a prevalence of more than 25%³. The diagnosis of AD is confirmed by 2 major histopathologic hallmarks: senile plaques, which are extracellular deposits of amyloid β (Aβ) peptides, and neurofibrillary tangles, which are somatic inclusions of the microtubule-associated protein tau.

The amyloid cascade hypothesis: structure and metabolic processing of the Amyloid β Precursor Protein (APP)

The role of Amyloid β (Aβ) peptide aggregation and deposition in AD pathogenesis is widely accepted. These toxic peptides are produced by the metabolic processing of the APP protein. APP is an integral membrane protein expressed in many tissues and concentrated in the synapses of neurons. APP proteolysis generates Aβ peptides containing 37 to 49 amino acids. The amyloid fibril (also called amyloid bodies) form of these peptides is the primary component of amyloid plaques found in the brains of AD patients.

Genetic alterations in APP suffice to cause early-onset Familial Alzheimer's Disease (FAD). Abnormalities induced by aggregated Aβ peptides have been linked to synaptic and neuritic degeneration. This is consistent with the “dying-back” pattern of degeneration that characterizes neurons affected in AD.

Amyloid plaque formation is a central pathological feature of AD. These plaques largely consist of Aβ peptides⁴. The Aβ peptides are formed through sequential proteolytic processing of the APP protein by β-secretase and γ-secretase⁵ (FIG. 1). The γ-secretase complex includes presenilin-1 and presenilin-2. β-secretase, also called the beta-site amyloid precursor protein cleaving enzyme 1 (BACE1), was originally identified about two decades ago^{6,7}. It cleaves APP mostly at a unique site (located between amino acids 671 and 672). However, the γ-secretase complex cleaves the resulting carboxy-terminal fragment at several places, especially at positions 40 and 42. The cleavage by this enzyme leads to the formation of amyloid Aβ1-40 and Aβ1-42 peptides⁵. Alternative processing of APP by the α-secretase prevents the formation of Aβ peptides because the α-site is located within Aβ peptides. The neurotoxic potential of the Aβ peptides results from their biochemical properties that favor aggregation into insoluble oligomers and protofibrils. These oligomers and protofibrils accumulate into senile and neuritic plaques. These plaques, along with a reduction of Aβ clearance from the brain, lead to the extracellular accumulation of Aβ into amyloid bodies. This leads to activation of neurotoxic cascades and ultimately to cytoskeletal changes, neuronal dysfunction and cellular death.

The amyloid cascade hypothesis is based mostly on findings from in vitro and in vivo studies, and is further strengthened by the discovery of genetic mutations associated with early-onset, familial AD. Familial ADs (FADs) are severe forms of the disease, in which massive intra-cerebral amyloidogenesis occurs prematurely as a consequence of mutations all affecting APP metabolism (i.e., mutations in the APP gene in chromosome 21, and in presenilin 1 and 2 (PSEN-1 and PSEN-2) genes in chromosomes 14 and 1 respectively). Currently, two proteins are deemed intimately involved in the clearance of Aβ peptides from the brain: apolipoprotein E (APOE) and the insulin-degrading enzyme (IDE). Disadvantageous genetic polymorphisms (such as the E4 allele of APOE) and pathological conditions related to abnormal IDE homeostasis (e.g., diabetes mellitus) also favor the amyloidogenic cleavage of APP and/or decrease the Aβ clearance from the brain. This facilitates the accumulation of Aβ in the neural tissues and promote downstream effects of the amyloid cascade⁸.

There is thus a need for methods and products to reduce the production of toxic Aβ peptides, and for the prevention and treatment of related conditions such as AD and age-related cognitive decline.

The present description refers to a number of documents and sequence database entries, the content of which is herein incorporated by reference in their entirety.

### SUMMARY OF THE DISCLOSURE

The present disclosure relates to the introduction of modifications in the Amyloid β Precursor Protein (APP) gene via base or prime editing using the CRISPR/CAS9 system, as well as to uses thereof, for example to decrease the production of amyloidogenic Aβ peptides, and to prevent or treat Alzheimer's disease and/or age-related cognitive decline.

More specifically, in accordance with aspects and embodiments of the present disclosure, there are provided the following items:

1. A method of decreasing Amyloid Precursor Protein (APP) processing into amyloidogenic Aβ peptide by a cell or the aggregation of the Aβ peptide, comprising providing said cell with:
a. a1) at least one guide RNA comprising a seed region of at least 18 consecutive nucleotides of a target sequence in an endogenous APP-encoding polynucleotide sequence present in said cell, wherein the target sequence of the guide RNA guide sequence is contiguous to a protospacer adjacent motif (PAM) in the endogenous APP gene polynucleotide sequence and wherein said PAM is recognized by a ribonucleoprotein complex comprising a Cas9 nickase; or
   a2) a vector comprising a nucleic acid sequence corresponding to the guide RNA for expressing the guide RNA; and
b. b1) a Cas nickase-deaminase fusion protein comprising (i) a Cas nickase polypeptide domain and (ii) a cytidine deaminase polypeptide domain or an adenosine deaminase polypeptide domain; or
   b2) a vector comprising a nucleic acid sequence encoding the Cas nickase-deaminase fusion protein, for expression of the Cas nickase-deaminase fusion protein; and
wherein at least one modification is introduced into the target sequence of the APP-encoding polynucleotide, wherein the at least one modification comprises modification of a cytidine into a thymidine or an adenosine into an inosine, and wherein the at least one modification decreases the processing of APP into amyloidogenic Aβ peptide by the cell or the aggregation of the Aβ peptide;
and wherein if the APP-encoding nucleotide is mRNA and the at least one modification comprises deamination of an adenosine into an inosine, the gRNA further comprises a cytidine mismatch with the adenosine to be deaminated to an inosine in the target sequence.
   2. The method of item 1, wherein said Cas nickase polypeptide domain comprises a SpCas9 nickase, a SaCas9 nickase, a CjCas9 nickase, ScCas9 nickase, CasX nickase, CasY nickase, Cpf1 nickase, eSaCas9 nickase eSpCas9 nickase or a HiFi Cas9 nickase.
   3. The method of item 1, wherein said Cas nickase-deaminase fusion protein is SpCas9n VQR BE3, SpCas9n EQR BE3, SaCas9n-KKH BE3, YE1-SpCas9n VQR BE3, YE1-SpCas9n EQR BE3, YE1-SaCas9n-KKH BE3, SpCas9n VQR BE4, SpCas9n EQR BE4, SaCas9n-KKH BE4, Target-AID SpCas9n VQR, Target-AID SpCas9n EQR, Target-AID SaCas9n-KKH or Target-AID-BE3.
   4. The method of any one of items 1 to 3, wherein the nucleic acid sequence corresponding to the guide RNA and the nucleic acid sequence encoding the Cas nickase-deaminase fusion protein are comprised in the same vector.
   5. The method of any one of items 1 to 4, wherein the at least one modification is located in intron 15, exon 16, intron 16, exon 17 and/or intron 17 of the APP-encoding polynucleotide sequence.
   6. The method of any one of items 1 to 5, wherein said at least one modification results in at least one amino acid substitution in the region defined by amino acid positions 656 to 688 in the APP protein, wherein said amino acid positions are with respect to the APP protein sequence as set forth in SEQ ID NO: 2 (NG_007376.1; FIG. 3).
   7. The method of item 6, wherein said at least one amino acid substitution corresponds to at least one substitution at amino acid position 670, 671, 673, 678, 682, 684, 692, 693, 694, 704, 711, 712, 713, 714, 715 and/or 684 of the APP protein.
   8. The method of item 7, wherein the at least one amino acid substitution introduces a threonine at position 673.
   9. The method of item 7, wherein the at least one amino acid substitution introduces of a valine at position 673.
   10. The method of item 7, wherein the at least one amino acid substitution introduces of an arginine at position 684.
   11. The method of any one of items 5-10, wherein said at least one modification in APP gene or mRNA results in a modification of one or more amino acids recognized by an α, β, or γ secretase in the APP protein encoded by said APP polynucleotide gene or mRNA sequence present in said cell.
   12. The method of any one of items 5-10, wherein said at least one modification corrects a mutation associated with an increased risk of developing Alzheimer's disease present in said endogenous APP polynucleotide gene sequence present in said cell.
   13. The method of any one of items 5-10, wherein said at least one modification results in a mutation which reduces risk of developing Alzheimer's disease by reducing the formation or the aggregation of Amyloidogenic peptides.
   14. The method of any one of items 1-13, wherein said cell is from a subject having at least one family member which has been diagnosed with Alzheimer's disease.
   15. The method of any one of items 1-13, wherein said cell is from a subject having at least one mutation associated with early onset Alzheimer's disease.
   16. The method of any one of items 1-13, wherein said cell is from a subject having a chance of developing a sporadic form Alzheimer's disease.
   17. The method of any one of items 1 to 16, wherein the guide RNA (guide RNA) sequence comprises at least 20 contiguous nucleotides of the target sequence in the endogenous APP polynucleotide gene or mRNA.
   18. The method of any one of items 1 to 17, wherein the PAM is MA, AAG, ACA, ATG, CC, CAA, CCN, GM, GAT, GGA, GGC, GGT, GTA, GTC, GTT, TCN, NG, NGG, NNG, NAG, NGA, NNAGAA, NNGAAT, NNGAGT, NNGGAT, NNGGGT, NNNAAT NNNAGT, NNNGAT, NNNGCA, NNNGGT, NNNNACA, NNNNACAC, NNNNATAC, NNNNGAAT, NNNNGATT, NNNNGCAC, NNNNGTAC, NNNNGTAT, NNNNGTTT, TGA, TGC, TGT, TTA, TTC, TTT, TTTA or a TTTC sequence.
   19. The method of any one of items 1 to 18, wherein the APP-encoding polynucleotide is an APP gene polynucleotide or an APP mRNA.
   20. The method of any one of items 1 to 19, wherein the target sequence of said guide RNA is located in an exon or an intron of the APP-encoding polynucleotide.
   21. The method of any one of items 1-20, wherein the target sequence is located between nucleotides 270798 and 294050 of the APP gene (SEQ ID NO: 1; ACCESSION NG_007376.1).
   22. The method of any one of items 1-21, wherein said at least one guide RNA comprises the following nucleic acid sequence:

SpCas9 VQR guide RNAs for A673T mutation:
(SEQ ID NO: 138)
AUUCUGCAUCCAUCUUCACUUC
SaCas9 guide RNAs for A673T mutation:
(SEQ ID NO: 139)
AUUCUGCAUCCAUCUUCACUUC
Target AID_SpCas9n guide RNA for A673V mutation:
(SEQ ID NO: 140)
GCAGAAUUCCGACAUGACUC
SpCas9 guide RNA for H684R mutation:
(SEQ ID NO: 141)
UGAAGUUCAUCAUCAAAAAU
SaCas9 KKH guide RNA for H684R mutation:
(SEQ ID NO: 142)
CAUCAUCAAAAAUUGGUACG
SpCas9 for ABE6.3, ABE7.8, ABE7.9 and
ABE7.10 for the H684R mutation in the DNA
(SEQ ID NO: 143)
UGAAGUUCAUCAUCAAAAAUGUUUAAGAGCUAUGCUGG
AAACAGCAUAGCAAGUUUAAAUAAGGCUAGUCCGUUAU
CAACUUGAAAAAGUGGCACCGAGUCGGUGC
Adenosine deaminase fused with SaCas9KKH
for the H684R mutation in the DNA
(SEQ ID NO: 144)
CAUCAUCAAAAAUUGGUACGGUUUUAGUACUCUGGAAACAG
AAUCUACUAAAACAAGGCAAAAUGCCGUGUUUAUCUCGUCA
ACUUGUUGGCGAGA
SpCas13b-ADAR2_{DD} (REPAIRv2) for the
H684R mutation in the mRNA
(SEQ ID NO: 145)
GAUGACGAACUUCAUAUCCUGAGUCAUGUCGGAAUUCUGCA
UCCAUCUUC
SpCas13b-ADAR2_{DD} (REPAIRv2) for the H684R
mutation in the mRNA
(SEQ ID NO: 146)
ACCAAUUUUUGAUGACGAACUUCAUAUCCUGAGUCAUGUCGGAAUUC
UGC
ADAR2 gRNA for H684R mutation:
(SEQ ID NO: 147)
5′GUGGAAUAGUAUAACAAUAUGCUAAAUGUUGUUAUAGUAUCCCAC
UUUUUGAUGACGAACUUC3′
wherein the underlined sequence is complementary
to the targeted sequence in the APP mRNA, and the
bold C is a mismatch for the nucleotide A to be
changed in a G in the APP mRNA.
REPAIRv2 gRNA for H684R mutation:
(SEQ ID NO: 148)
5′ACCAAUUUUUGAUGACGAACUUCAUAUCCUGAGUCAU
GUUGUGGAAGGUCCAGUUUUGGGGGCUAUUACAACA3′
wherein the underlined sequence is complementary
to the targeted sequence in the APP mRNA, and the
bold C is a mismatch for the nucleotide A to be
changed in a G in the APP mRNA.

23. The method of any one of items 1 to 22, wherein the Cas nickase-deaminase fusion protein further comprises a protein transduction domain (PTD).
24. The method of any one of items 1 to 22, wherein the Cas nickase-deaminase fusion protein further comprises a nuclear localization signal (NLS).
25. The method of any one of items 1 to 24, wherein the vector is a viral vector, a virus like particle (VLP), an extra-cellular vesicle or an exosome.
26. The method of item 25, wherein said viral vector is a lentiviral vector, Adeno-Associated viral vector, adenovirus viral vector or herpes virus viral vector.
27. A guide RNA as defined in any one of items 1 to 22.
28. An isolated nucleic acid comprising a nucleotide sequence corresponding to the guide RNA of item 27 for expressing the guide RNA.
29. An isolated nucleic acid encoding the Cas nickase-deaminase fusion protein defined in any one of items 1 to 24.
30. A vector comprising the isolated nucleic acid of item 28 and/or the isolated nucleic acid of item 29.
31. The vector of item 30, wherein the vector is a viral vector, a virus like particle (VLP), an extra-cellular vesicle or an exosome.
32. The vector of item 31, wherein the viral vector is a lentiviral vector, Adeno-Associated viral vector, adenovirus viral vector or herpes virus viral vector.
33. A system comprising:
i) the guide RNA of item 27,
ii) the Cas nickase-deaminase fusion protein defined in any one of items 1 to 24,
iii) the isolated nucleic acid of item 28 or 29,
iv) the vector of any one of items 30 to 32, or
v) any combination of (i) to (iv).
   34. A composition comprising a biologically acceptable carrier or a pharmaceutically acceptable carrier and:
i) the guide RNA of item 27,
ii) the Cas nickase-deaminase fusion protein defined in any one of items 1 to 24,
iii) the isolated nucleic acid of item 28 or 29,
iv) the vector of any one of items 30 to 32, or
v) any combination of (i) to (iv).
   35. The guide RNA of item 27, the Cas nickase-deaminase fusion protein defined in any one of items 1 to 24, the isolated nucleic acid of item 28 or 29, the vector of any one of items 30 to 32, the system of item 33, and/or the composition of item 34 for use in decreasing Amyloid Precursor Protein (APP) processing into amyloidogenic Aβ peptide by a cell or the aggregation of the Aβ peptide.
   36. The guide RNA of item 27, the Cas nickase-deaminase fusion protein defined in any one of items 1 to 24, the isolated nucleic acid of item 28 or 29, the vector of any one of items 30 to 32, the system of item 33, and/or the composition of item 34 for the preparation of a medicament for decreasing Amyloid Precursor Protein (APP) processing into amyloidogenic Aβ peptide by a cell or the aggregation of the Aβ peptide.
   37. The guide RNA, Cas nickase-deaminase fusion protein, isolated nucleic acid, vector, system, and/or composition for use of item 35 or 36, wherein the cell is from a subject having at least one family member which has been diagnosed with Alzheimer's disease.
   38. The guide RNA, Cas nickase-deaminase fusion protein, isolated nucleic acid, vector, system, and/or composition for use of item 35 or 36, wherein the cell is from a subject having at least one mutation associated with early onset Alzheimer's disease.
   39. The guide RNA of item 27, the Cas nickase-deaminase fusion protein defined in any one of items 1 to 24, the isolated nucleic acid of item 28 or 29, the vector of any one of items 30 to 32, the system of item 33, and/or the composition of item 34 for use in treating or preventing Alzheimer's disease or age-related cognitive decline in a subject in need thereof.
   40. The guide RNA of item 27, the Cas nickase-deaminase fusion protein defined in any one of items 1 to 24, the isolated nucleic acid of item 28 or 29, the vector of any one of items 30 to 32, the system of item 33, and/or the composition of item 34 for use in the preparation of a medicament for treating or preventing Alzheimer's disease or age-related cognitive decline in a subject in need thereof.
   41. The guide RNA, Cas nickase-deaminase fusion protein, isolated nucleic acid, vector, system, and/or composition for use of item 39 or 40, wherein the subject in need thereof is a subject having at least one family member (e.g., a mother, father, brother, sister or child), who has been diagnosed with Alzheimer's disease.
   42. Use of the guide RNA of item 27, the Cas nickase-deaminase fusion protein defined in any one of items 1 to 24, the isolated nucleic acid of item 28 or 29, the vector of any one of items 30 to 32, the system of item 33, and/or the composition of item 34 for decreasing Amyloid Precursor Protein (APP) processing into amyloidogenic Aβ peptide by a cell or the aggregation of the Aβ peptide.
   43. Use of the guide RNA of item 27, the Cas nickase-deaminase fusion protein defined in any one of items 1 to 24, the isolated nucleic acid of item 28 or 29, the vector of any one of items 30 to 32, the system of item 33, and/or the composition of item 34 for the preparation of a medicament for decreasing Amyloid Precursor Protein (APP) processing into amyloidogenic Aβ peptide by a cell or the aggregation of the Aβ peptide.
   44. The use of item 42 or 43, wherein the cell is from a subject having at least one family member which has been diagnosed with Alzheimer's disease.
   45. The use of item 42 or 43, wherein the cell is from a subject having at least one mutation associated with early onset Alzheimer's disease.
   46. Use of the guide RNA of item 27, the Cas nickase-deaminase fusion protein defined in any one of items 1 to 24, the isolated nucleic acid of item 28 or 29, the vector of any one of items 30 to 32, the system of item 33, and/or the composition of item 34, for treating or preventing Alzheimer's disease or age-related cognitive decline in a subject in need thereof.
   47. Use of the guide RNA of item 27, the Cas nickase-deaminase fusion protein defined in any one of items 1 to 24, the isolated nucleic acid of item 28 or 29, the vector of any one of items 30 to 32, the system of item 33, and/or the composition of item 34, for the preparation of a medicament for treating or preventing Alzheimer's disease or age-related cognitive decline in a subject in need thereof.
   48. The use of item 46 or 47, wherein said subject in need thereof is a subject having at least one family member (e.g., a mother, father, brother, sister or child), who has been diagnosed with Alzheimer's disease.
   49. The use of item 46 or 47, wherein said subject in need thereof is a subject having at least one mutation associated with early onset Alzheimer's disease.
   50. A method of decreasing Amyloid Precursor Protein (APP) processing into amyloidogenic Aβ peptide by a cell or the aggregation of the Aβ peptide, comprising introducing into the cell or contacting the cell with the guide RNA of item 27, the Cas nickase-deaminase fusion protein defined in any one of items 1 to 24, the isolated nucleic acid of item 28 or 29, the vector of any one of items 30 to 32, the system of item 33, and/or the composition of item 34.
   51. A method of decreasing Amyloid Precursor Protein (APP) processing into amyloidogenic Aβ peptide by a cell or the aggregation of the Aβ peptide, comprising providing said cell with:
a. a1) at least one pegRNA comprising a seed region of at least 18 consecutive nucleotides of a target sequence in an endogenous APP-encoding polynucleotide sequence present in said cell, wherein the target sequence of the pegRNA guide sequence is contiguous to a protospacer adjacent motif (PAM) in the endogenous APP gene polynucleotide sequence and wherein said PAM is recognized by a ribonucleoprotein complex comprising a Cas9 nickase; or
   a2) a vector comprising a nucleic acid sequence corresponding to the pegRNA for expressing the pegRNA; and
b. b1) a Cas nickase-reverse transcriptase fusion protein comprising (i) a Cas nickase polypeptide domain and (ii) a reverse transcriptase polypeptide domain; or
   b2) a vector comprising a nucleic acid sequence encoding the Cas nickase-reverse transcriptase fusion protein, for expression of the Cas nickase-reverse transcriptase fusion protein; and
wherein at least one modification is introduced into the target sequence of the APP-encoding polynucleotide, wherein the at least one modification decreases the processing of APP into amyloidogenic Aβ peptide by the cell or the aggregation of the Aβ peptide.
   52. The method of item 51, wherein said Cas nickase polypeptide domain comprises a SpCas9 nickase, a SaCas9 nickase, a CjCas9 nickase, ScCas9 nickase, CasX nickase, CasY nickase, Cpf1 nickase, eSaCas9 nickase eSpCas9 nickase, a HiFi Cas9 nickase, or a Cas9 H840A nickase.
   53. The method of item 1, wherein said reverse transcriptase domain comprises an M-MLV reverse transcriptase.
   54. The method of any one of items 51 to 53, wherein the nucleic acid sequence corresponding to the guide RNA or pegRNA and the nucleic acid sequence encoding the Cas nickase-reverse transcriptase fusion protein are comprised in the same vector.
   55. The method of any one of items 51 to 54, wherein the at least one modification is located in intron 15, exon 16, intron 16, exon 17 and/or intron 17 of the APP-encoding polynucleotide sequence.
   56. The method of any one of items 51 to 55, wherein said at least one modification results in at least one amino acid substitution in the region defined by amino acid positions 656 to 688 in the APP protein, wherein said amino acid positions are with respect to the APP protein sequence as set forth in SEQ ID NO: 2 (NG_007376.1; FIG. 3).
   57. The method of item 56, wherein said at least one amino acid substitution corresponds to at least one substitution at amino acid position 670, 671, 673, 678, 682, 684, 692, 693, 694, 704, 711, 712, 713, 714, 715 and/or 684 of the APP protein.
   58. The method of item 57, wherein the at least one amino acid substitution introduces a threonine at position 673.
   59. The method of item 57, wherein the at least one amino acid substitution introduces of a valine at position 673.
   60. The method of item 57, wherein the at least one amino acid substitution introduces of an arginine at position 684.
   61. The method of any one of items 55-60, wherein said at least one modification in APP gene or mRNA results in a modification of one or more amino acids recognized by an α, β, or γ secretase in the APP protein encoded by said APP polynucleotide gene or mRNA sequence present in said cell.
   62. The method of any one of items 55-60, wherein said at least one modification corrects a mutation associated with an increased risk of developing Alzheimer's disease present in said endogenous APP polynucleotide gene sequence present in said cell.
   63. The method of any one of items 55-60, wherein said at least one modification results in a mutation which reduces risk of developing Alzheimer's disease by reducing the formation or the aggregation of Amyloidogenic peptides.
   64. The method of any one of items 51-63, wherein said cell is from a subject having at least one family member which has been diagnosed with Alzheimer's disease.
   65. The method of any one of items 51-63, wherein said cell is from a subject having at least one mutation associated with early onset Alzheimer's disease.
   66. The method of any one of items 51-63, wherein said cell is from a subject having a chance of developing a sporadic form Alzheimer's disease.
   67. The method of any one of items 51 to 66, wherein the pegRNA guide sequence comprises at least 20 contiguous nucleotides of the target sequence in the endogenous APP polynucleotide gene or mRNA.
   68. The method of any one of items 51 to 67, wherein the PAM is AAA, MG, ACA, ATG, CC, CAA, CCN, GAA, GAT, GGA, GGC, GGT, GTA, GTC, GTT, TCN, NG, NGG, NNG, NAG, NGA, NNAGAA, NNGAAT, NNGAGT, NNGGAT, NNGGGT, NNNAAT NNNAGT, NNNGAT, NNNGCA, NNNGGT, NNNNACA, NNNNACAC, NNNNATAC, NNNNGAAT, NNNNGATT, NNNNGCAC, NNNNGTAC, NNNNGTAT, NNNNGTTT, TGA, TGC, TGT, TTA, TTC, TTT, TTTA or a TTTC sequence.
   69. The method of any one of items 51 to 68, wherein the APP-encoding polynucleotide is an APP gene polynucleotide or an APP mRNA.
   70. The method of any one of items 51 to 69, wherein the target sequence of said pegRNA is located in an exon or an intron of the APP-encoding polynucleotide.
   71. The method of any one of items 51-70, wherein the target sequence is located between nucleotides 270798 and 294050 of the APP gene (SEQ ID NO: 1; ACCESSION NG_007376.1).
   72. The method of any one of items 51-71, wherein said at least one pegRNA comprises the following nucleic acid sequence:

SpCas9 VQR guide RNA for A673T mutation:
(SEQ ID NO: 138)
AUUCUGCAUCCAUCUUCACUUC
SaCas9 guide RNA for A673T mutation:
(SEQ ID NO: 139)
AUUCUGCAUCCAUCUUCACUUC
Target AID_SpCas9n guide RNA for A673V mutation:
(SEQ ID NO: 140)
GCAGAAUUCCGACAUGACUC
SpCas9 guide RNA for H684R mutation:
(SEQ ID NO: 141)
UGAAGUUCAUCAUCAAAAAU
SaCas9 KKH guide RNA for H684R mutation:
(SEQ ID NO: 142)
CAUCAUCAAAAAUUGGUACG
SpCas9 for ABE6.3, ABE7.8, ABE7.9 and
ABE7.10 for the H684R mutation in the DNA
(SEQ ID NO: 143)
UGAAGUUCAUCAUCAAAAAUGUUUAAGAGCUAUGCUGG
AAACAGCAUAGCAAGUUUAAAUAAGGCUAGUCCGUUAU
CAACUUGAAAAAGUGGCACCGAGUCGGUGC
Adenosine deaminase fused with SaCas9KKH
for the H684R mutation in the DNA
(SEQ ID NO: 144)
CAUCAUCAAAAAUUGGUACGGUUUUAGUACUCUGGAAA
CAGAAUCUACUAAAACAAGGCAAAAUGCCGUGUUUAUC
UCGUCAACUUGUUGGCGAGA
SpCas13b-ADAR2_{DD} (REPAIRv2) for the
H684R mutation in the mRNA
(SEQ ID NO: 145)
GAUGACGAACUUCAUAUCCUGAGUCAUGUCGGAAUUCU
GCAUCCAUCUUC
SpCas13b-ADAR2_{DD} (REPAIRv2) for the
H684R mutation in the mRNA
(SEQ ID NO: 146)
ACCAAUUUUUGAUGACGAACUUCAUAUCCUGAGUCAUG
UCGGAAUUCUGC
ADAR2 gRNA for H684R mutation:
(SEQ ID NO: 147)
5′GUGGAAUAGUAUAACAAUAUGCUAAAUGUUGUUA
UAGUAUCCCACUUUUUGAUGACGAACUUC3′
wherein the underlined sequence is complementary to
the targeted sequence in the APP mRNA, and the
bold C is a mismatch for the nucleotide A to be
changed in a G in the APP mRNA.
REPAIRv2 gRNA for H684R mutation:
(SEQ ID NO: 148)
5′ACCAAUUUUUGAUGACGAACUUCAUAUCCUGAG
UCAUGUUGUGGAAGGUCCAGUUUUGGGGGCU
AUUACAACA3′
wherein the underlined sequence is complementary
to the targeted sequence in the APP mRNA, and the
bold C is a mismatch for the nucleotide A to be
changed in a G in the APP mRNA.

73. The method of any one of items 51 to 72, wherein the Cas nickase-reverse transcriptase fusion protein further comprises a protein transduction domain (PTD).
74. The method of any one of items 51 to 72, wherein the Cas nickase-reverse transcriptase fusion protein further comprises a nuclear localization signal (NLS).
75. The method of any one of items 51 to 74, wherein the vector is a viral vector, a virus like particle (VLP), an extra-cellular vesicle or an exosome.
76. The method of item 75, wherein said viral vector is a lentiviral vector, Adeno-Associated viral vector, adenovirus viral vector or herpes virus viral vector.
77. A pegRNA as defined in any one of items 51 to 72.
78. An isolated nucleic acid comprising a nucleotide sequence corresponding to the pegRNA of item 77 for expressing the pegRNA.
79. An isolated nucleic acid encoding the Cas nickase-reverse transcriptase fusion protein defined in any one of items 51 to 74.
80. A vector comprising the isolated nucleic acid of item 78 and/or the isolated nucleic acid of item 79.
81. The vector of item 80, wherein the vector is a viral vector, a virus like particle (VLP), an extra-cellular vesicle or an exosome.
82. The vector of item 81, wherein the viral vector is a lentiviral vector, Adeno-Associated viral vector, adenovirus viral vector or herpes virus viral vector.
83. A system comprising:
vi) the pegRNA of item 77,
vii) the Cas nickase-reverse transcriptase fusion protein defined in any one of items 51 to 74,
viii) the isolated nucleic acid of item 78 or 79,
ix) the vector of any one of items 80 to 82, or
x) any combination of (i) to (iv).
   84. A composition comprising a biologically acceptable carrier or a pharmaceutically acceptable carrier and:
vi) the pegRNA of item 77,
vii) the Cas nickase-deaminase or Cas nickase-reverse transcriptase fusion protein defined in any one of items 51 to 74,
viii) the isolated nucleic acid of item 78 or 79,
ix) the vector of any one of items 80 to 82, or
x) any combination of (i) to (iv).
   85. The pegRNA of item 77, the Cas nickase-reverse transcriptase fusion protein defined in any one of items 51 to 74, the isolated nucleic acid of item 78 or 79, the vector of any one of items 80 to 82, the system of item 83, and/or the composition of item 84 for use in decreasing Amyloid Precursor Protein (APP) processing into amyloidogenic Aβ peptide by a cell or the aggregation of the Aβ peptide.
   86. The pegRNA of item 77, the Cas nickase-reverse transcriptase fusion protein defined in any one of items 51 to 74, the isolated nucleic acid of item 78 or 79, the vector of any one of items 80 to 82, the system of item 83, and/or the composition of item 84 for the preparation of a medicament for decreasing Amyloid Precursor Protein (APP) processing into amyloidogenic Aβ peptide by a cell or the aggregation of the Aβ peptide.
   87. The pegRNA, Cas nickase-reverse transcriptase fusion protein, isolated nucleic acid, vector, system, and/or composition for use of item 85 or 86, wherein the cell is from a subject having at least one family member which has been diagnosed with Alzheimer's disease.
   88. The pegRNA, Cas nickase-reverse transcriptase fusion protein, isolated nucleic acid, vector, system, and/or composition for use of item 85 or 86, wherein the cell is from a subject having at least one mutation associated with early onset Alzheimer's disease.
   89. The pegRNA of item 77, the Cas nickase-reverse transcriptase fusion protein defined in any one of items 51 to 74, the isolated nucleic acid of item 78 or 79, the vector of any one of items 80 to 82, the system of item 83, and/or the composition of item 84 for use in treating or preventing Alzheimer's disease or age-related cognitive decline in a subject in need thereof.
   90. The pegRNA of item 77, the Cas nickase-reverse transcriptase fusion protein defined in any one of items 51 to 74, the isolated nucleic acid of item 78 or 79, the vector of any one of items 80 to 82, the system of item 83, and/or the composition of item 84 for use in the preparation of a medicament for treating or preventing Alzheimer's disease or age-related cognitive decline in a subject in need thereof.
   91. The pegRNA, Cas nickase-reverse transcriptase fusion protein, isolated nucleic acid, vector, system, and/or composition for use of item 89 or 90, wherein the subject in need thereof is a subject having at least one family member (e.g., a mother, father, brother, sister or child), who has been diagnosed with Alzheimer's disease.
   92. Use of the pegRNA of item 77, the Cas nickase-reverse transcriptase fusion protein defined in any one of items 51 to 74, the isolated nucleic acid of item 78 or 89, the vector of any one of items 80 to 82, the system of item 83, and/or the composition of item 84 for decreasing Amyloid Precursor Protein (APP) processing into amyloidogenic Aβ peptide by a cell or the aggregation of the Aβ peptide.
   93. Use of the pegRNA of item 77, the Cas nickase-reverse transcriptase fusion protein defined in any one of items 51 to 74, the isolated nucleic acid of item 78 or 89, the vector of any one of items 80 to 82, the system of item 83, and/or the composition of item 84 for the preparation of a medicament for decreasing Amyloid Precursor Protein (APP) processing into amyloidogenic Aβ peptide by a cell or the aggregation of the Aβ peptide.
   94. The use of item 92 or 93, wherein the cell is from a subject having at least one family member which has been diagnosed with Alzheimer's disease.
   95. The use of item 92 or 93, wherein the cell is from a subject having at least one mutation associated with early onset Alzheimer's disease.
   96. Use of the pegRNA of item 77, the Cas nickase-reverse transcriptase fusion protein defined in any one of items 51 to 74, the isolated nucleic acid of item 78 or 89, the vector of any one of items 80 to 82, the system of item 83, and/or the composition of item 84, for treating or preventing Alzheimer's disease or age-related cognitive decline in a subject in need thereof.
   97. Use of the pegRNA of item 77, the Cas nickase-reverse transcriptase fusion protein defined in any one of items 51 to 74, the isolated nucleic acid of item 78 or 89, the vector of any one of items 80 to 82, the system of item 83, and/or the composition of item 84, for the preparation of a medicament for treating or preventing Alzheimer's disease or age-related cognitive decline in a subject in need thereof.
   98. The use of item 96 or 97, wherein said subject in need thereof is a subject having at least one family member (e.g., a mother, father, brother, sister or child), who has been diagnosed with Alzheimer's disease.
   99. The use of item 96 or 97, wherein said subject in need thereof is a subject having at least one mutation associated with early onset Alzheimer's disease.
   100. A method of decreasing Amyloid Precursor Protein (APP) processing into amyloidogenic Aβ peptide by a cell or the aggregation of the Aβ peptide, comprising introducing into the cell or contacting the cell with the pegRNA of item 77, the Cas nickase-reverse transcriptase fusion protein defined in any one of items 51 to 74, the isolated nucleic acid of item 78 or 89, the vector of any one of items 80 to 82, the system of item 83, and/or the composition of item 84.

Other objects, advantages and features of the present disclosure will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows pathways for metabolic processing of Amyloid Precursor Protein (APP). The figure illustrates the two most prominent metabolic routes and the APP proteolytic derivatives. APP is the substrate for either α-, β- and/or γ-secretase. Thus, the two pathways (non-amyloidogenic (also called amyloidolytic) and the amyloidogenic) are mutually exclusive. 6-site APP-cleaving enzyme 1 (BACE1) is the only known aspartyl protease with β-secretase activity. The fragments C83 and C99 (also known as α- and β-carboxy-terminal fragments) are substrates for the γ-secretase, a protein complex that includes at least 4 different proteins: anterior pharynx defective 1, nicastrin, presenilin 1 (PS-1) or presenilin 2 (PS-2), and presenilin enhancer-2. AICD is the intra-cytoplasmic domain of APP. The figure was adapted from E. A. Bignante et al.⁹.

FIG. 2 is a schematic representation of the domain structure of amyloid precursor protein (APP). The E1 domain contains the Growth Factor Like Domain (GFLD) and the Copper-Binding Domain (CuBD). The acidic domain (AcD) and the Kunitz-type inhibitory domain (Kunitz protease inhibitor [KPI], not present in neurons) bridge the E1 and E2 domains. The E2 domain contains 2 coiled-coil substructures connected through a continuous helix. The amino acid sequence (SEQ ID NO: 45) of amyloid β (Aβ)/trans-membrane/intracellular (intra-cytoplasmic domain of APP [AICD]) domains is shown in the lower part of the figure. Asterisks denote amino acids substituted in familial forms of AD and APP variants. Arrows indicate cleavage sites for both secretases and caspases. Binding motifs for heterotrimeric Go/s proteins (Go/s-BM) and scaffolding proteins (SP-BM; e.g., Fe65, Mint/X11-family proteins, Dab1, c-Jun N-terminal kinase) are underlined. The Aβ sequence is also underlined. From E. A. Bignante et al.⁹.

FIG. 3 shows the complete amino acid sequence of the human Amyloid β Precursor Protein (APP) gene (SEQ ID NO: 2; NCBI Reference Sequence: NG_007376.1; nucleotide sequence shown in SEQ ID NO: 1). The bold A is alanine at position 673 to be mutated to a threonine (T) or a valine (V) to protect from Alzheimer's disease. The bold H is histidine at position 684 to be mutated to an arginine (R) to protect from Alzheimer's disease.

FIG. 4 illustrates the structure of BE3, CDA1-BE3, and Target-AID-BE3. From Komor et al.¹⁰.

FIG. 5 illustrates part of APP exon 16. A illustrates the antisense strand (SEQ ID NO: 52). There are 5 cytidines close to one another, which can be deaminated by SpCas9 base editing. The position of these 5 cytidines in the wild type exon 16 of the APP gene and the corresponding amino acid codons (from E665 to F675). B) Deamination of all 5 cytidines (C1-05) into thymidines is shown, which changes the codons and thus the resulting amino acid, except for cytidine 5 for which the change into a thymidine does not change the resulting amino acid, which remains a lysine (K). Sense strand, SEQ ID NO: 46; antisense strand, SEQ ID NO: 47; amino acid sequence SEQ ID NO: 48. C) The deamination of only cytidine 2 in the antisense codon for alanine changes the codon in position 673 into a threonine codon. The cut site of BACE1 between methionine 671 and aspartic acid 672 is indicated. Sense strand, SEQ ID NO: 49; antisense strand, SEQ ID NO: 50; amino acid sequence SEQ ID NO: 51.

FIG. 6 shows the percentages of deamination in HEK293T cells with one or two copies of guide RNA. Plasmids coding for the various cytidine deaminases and for one guide RNA (left side of the figure) or two guide RNAs (right side of the figure) were transfected into HEK293T cells. The DNA was extracted 3 days later, APP exon 16 was PCR amplified and analyzed by deep sequencing. The sequences were computer analyzed to establish the percentages of the cytidines 1 to 5 (FIG. 5; bars in each grouping correspond to C1-05 from left to right), which were changed into a thymidine with the different deaminases (BE3_SpCas9nVQR with a guide RNA targeting 20 nucleotides, BE3_SpCas9nVQR with a guide RNA targeting 21 nucleotides).

FIG. 7 shows the percentages of deamination in SH-SY5Y and HEK293T cells with two copies of guide RNA. Plasmids coding for various deaminase enzymes and two copies of the guide RNA were transfected in SH-SY5Y neuroblastomas (left side) and in HEK293T cells (right side). The guide RNAs were targeting either 19, 20, 21 or 22 nucleotides. The DNA was extracted 3 days later, APP exon 16 was PCR amplified and analyzed by deep sequencing. The sequences were computer analyzed to establish the percentages of the cytidines 1 to 5 (FIG. 5; bars in each grouping correspond to C1-05 from left to right), which were changed into a thymidine with different deaminases (Target-AID-SpCas9nEQR, Target-AID-SpCas9nVQR, BE3_SpCas9nVQR).

FIG. 8 shows the percentages of deamination in SH-SY5Y cells with two copies of guide RNA. Plasmids coding for various deaminases and a guide RNA were transfected in SH-SY5Y neuroblastomas. The guide RNAs were targeting either 19, 20, 21 or 22 nucleotides. The DNA was extracted 3 days post-transfection, APP exon 16 was PCR amplified and analyzed by deep sequencing. The sequences were computer analyzed to establish the percentages of the cytidines 1 to 5 (FIG. 5; bars in each grouping correspond to C1-05 from left to right), which were changed into a thymidine with two different deaminases (YE1-BE3_SpCas9nVQR and BE3_SpCas9nVQR).

FIG. 9 shows the percentages of deamination in SH-SY5Y cells with two copies of guide RNA. Plasmids coding for various deaminases and two copies of a guide RNA were transfected in SH-SY5Y neuroblastomas. The guide RNAs were targeting 19 nucleotides. The DNA was extracted 3 days later, APP exon 16 was PCR amplified and analyzed by deep sequencing. The sequences were computer analyzed to establish the percentages of the cytidines 1 to 5 (FIG. 5; bars in each grouping correspond to C1-05 from left to right), which were changed into a thymidine with two different deaminases (BE3_SpCas9nEQR, BE3_SpCas9nVQR, BE3_SaCas9nKKH). “Not treated” shows the results in cells that were not transfected.

FIG. 10 shows the percentages of deamination in SH-SY5Y cells with two copies of guide RNA. Plasmids coding for various deaminases and two copies of a guide RNA were transfected in SH-SY5Y neuroblastomas. The guide RNAs were targeting 17, 18, 19, 20 nucleotides. The DNA was extracted 3 days later, APP exon 16 was PCR amplified and analyzed by deep sequencing. The sequences were computer analyzed to establish the percentages of the cytidines 1 to 5 (FIG. 5; bars in each grouping correspond to C1-05 from left to right), which were changed into a thymidine with two different deaminases (Target-AID-SpCas9nVQR and BE3_SpCas9nVQR). The WT illustrates the results in cells that were not transfected.

FIG. 11 shows the percentages of deamination in HEK293T cells with two copies of guide RNA. Plasmids coding for the Target-AID-SpCas9nVQR enzyme and two copies of a guide RNA were transfected in HEK293T cells. The guide RNAs were targeting 19, 20, 21 and 22 nucleotides. The DNA was extracted 3 days later, APP exon 16 was PCR amplified and analyzed by deep sequencing. The sequences were computer analyzed to establish the percentages of the cytidines 1 to 5 (FIG. 5; bars in each grouping correspond to C1-05 from left to right), which were changed into a thymidine. More than 50% of the cytidines 1 and 2 (FIG. 5) were deaminated with 19 bp guide RNAs.

FIG. 12 shows the percentages of deamination in SH-SY5Y cells. SH-SY5Y cells were transfected with one APP plasmid containing a FAD mutation and with the Target-AID-SpCas9n_{VQR} plasmid containing two copies of the guide RNA targeting 19 nucleotides. The deep sequencing results showed a strong global deamination activity.

FIG. 13 shows the percentages of deamination in SH-SY5Y cells. SH-SY5Y cells were transfected with one APP plasmid containing a FAD mutation and with the Target-AID-SpCas9n_{VQR} plasmid containing two copies of the guide RNA targeting 19 nucleotides. Different deamination profiles were obtained with APP plasmids containing different FAD mutations. Bars in each grouping correspond to C1-05 from left to right. Indeed, C1 was not as frequently deaminated as C2. The ten best FAD mutations showed between 5.3-19.4% deamination of C2.

FIG. 14 shows the effect of deamination of C2 on Aß42 peptide levels in SH-SY5Y cells. SH-SY5Y cells containing the Target-AID-SpCas9n_{VQR} transgene and a sequence coding for guide RNA targeting 17 or 19 nucleotides were transfected with a plasmid containing the wild type APP gene alone (left side) or co-transfected with plasmids coding for the wild type APP gene and a guide RNA (right side) to permit the deamination of the C2 nucleotide and thus induce the A673T mutation. The induction of the A673T mutation reduced by 40% the concentration of Aß42 peptides in the APP WT-transfected cell supernatant, by 43% the concentration of Aß42 peptides in the APP NL/F/G-transfected cell supernatant and by 24% the concentration of Aß42 peptides in the APP V717I-transfected cell supernatant.

FIG. 15 illustrates the end of exon 16 and the beginning of intron 16 of the APP gene. The amino acids corresponding to each codon are illustrated at the bottom. The codons for alanine in position 673 and for histidine in position 684 are indicated. The positions of the NGAN PAM for the SpCas9nVQR permitting to test several cytidine deaminases (i.e., BE3_SpCas9nVQR, the YE1-BE3_SpCas9nVQR, BE4-Gam_SpCas9nVQR and the Target-AID_SpCas9nVQR) to deaminate the cytidine into a thymidine in the alanine codon (GCA) to produce the GTA codon for valine. The figure also illustrates the position of a NGG PAM and of a NNNAAT PAM, which permit to deaminate the adenosine into an inosine (equivalent to a guanine) modify the histidine codon (CAT) into a CGT codon for arginine with appropriate adenosine deaminases.

FIG. 16 illustrates the percentages of a cytidine deamination in an experiment aiming to modify the alanine (at position 673) codon (GCA) into a valine codon (GTA) by deaminating the cytidine no 1 (in position 2 of the protospacer sequence) into a thymidine. 27.82% of the C were changed into a T using the Target-AID_SpCas9n. A very low percentage (0.27%) of the second C in the protospacer motif were changed into a T. The other cytidine deaminases had very low effects. In a second experiment using the Target-AID_SpCas9n 18.48% of the first C were deaminated.

FIG. 17 illustrates the percentages of an adenosine deamination using ADAR2 to modify the histidine (at position 684) codon (CAT) into an arginine codon (CGT). Two different guide RNAs were used. For the first guide RNA the A to be deaminated was in position 35 and for the second guide RNA the A to be deaminated was in position 45. The guide RNA with the A in position 35 gave better results than the guide RNA with the A in position 45.

FIG. 18 illustrates the end of exon 16 and the beginning of intron 16 of the APP gene (sense sequence, SEQ ID NO: 39; antisense sequence, SEQ ID NO: 40; amino acid sequence, SEQ ID NO: 41). The amino acids corresponding to each codon are illustrated at the bottom. The codons for alanine in position 673 and for histidine in position 684 are indicated. The positions of the NGAN PAM for the SpCas9nVQR permitting to test several cytidine deaminases (i.e., BE3_SpCas9nVQR, the YE1-BE3_SpCas9nVQR, BE4-Gam_SpCas9nVQR and the Target-AID_SpCas9nVQR) to deaminate the cytidine into a thymidine in the alanine codon (GCA) to produce the GTA codon for valine. The figure also illustrates the position of a NGG PAM and of a NNNAAT PAM, which permit to deaminate the adenosine into an inosine (equivalent to a guanine) modify the histidine codon (CAT) into a CGT codon for arginine with appropriate adenosine deaminases . . . .

FIG. 19 illustrates the results of a cytidine deaminase experiment aiming to modify the alanine (at position 673) codon (GCA) into a valine codon (GTA) by deaminating the cytidine no 1 (in position 2 of the protospacer sequence) into a thymidine. 27.82% of the C were changed into a T using the Target-AID_SpCas9n. A very low percentage (0.27%) of the second C in the protospacer motif were changed into a T. The other cytidine deaminase has very low effects. In a second experiment using the Target-AID_SpCas9n 18.48% of the first C were deaminated.

FIG. 20 illustrates the results of an adenosine deaminase (ADAR2) experiment aiming to modify the histidine (at position 684) codon (CAT) into an arginine codon (CGT). Two different gRNAs were used. For the first gRNA the A to be deaminated was in position 35 and for the second gRNA the A to be deaminated was in position 45. The gRNA with the A in position 35 gave better results than the gRNA with the A in position 45.

FIG. 21 shows the nucleotide (SEQ ID NO: 42) and amino acid (SEQ ID NO: 43) sequences of exon 16 from APP (NCBI Reference sequence NG_007376.1). A673 and H684 are shown in bold.

FIG. 22 shows a partial sequence of the wild type APP gene (nucleotides 277141 to 279540 of NCBI NG_007376.1/GI: 166795291, SEQ ID NO: 44; full APP gene sequence in SEQ ID NO: 1) which includes a partial sequence of intron 15, followed by the complete sequence of exon 16 (nucleotides 278148-278248) and a partial sequence of intron 16.

### DESCRIPTION OF ILLUSTRATIVE EMBODIMENTS

### Definitions

The use of the terms “a” and “an” and “the” and similar referents in the context of describing the technology (especially in the context of the following claims) are to be construed to cover both the singular and the plural, unless otherwise indicated herein or clearly contradicted by context.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods or processes described herein can be performed in any suitable order unless otherwise indicated herein or otherwise clearly contradicted by context. Further, in embodiments various steps may be repeated, to increase recovery and purification.

The use of any and all examples, or exemplary language (e.g., “such as”) provided herein, is intended merely to better illuminate the disclosure and does not pose a limitation on the scope of the disclosure unless otherwise claimed.

No language in the specification should be construed as indicating any non-claimed element as essential to the practice of the disclosure.

As used herein when referring to numerical values or percentages, the term “about” has its ordinary meaning, and includes variations due to the methods used to determine the values or percentages, statistical variance and human error. Moreover, each numerical parameter in this application should at least be construed in light of the number of reported significant digits and by applying ordinary rounding techniques. In embodiments, it may mean plus or minus 10% of the numerical value qualified.

Unless otherwise defined, all technical and scientific terms used herein have the same meaning as commonly understood by one of ordinary skill in the art to which this disclosure belongs.

It is to be understood that the present disclosure is not limited to the particular embodiments described below, as variations of these embodiments may be made and still fall within the scope of the present disclosure. It is also to be understood that the terminology employed is for the purpose of describing particular embodiments; and is not intended to be limiting.

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this disclosure pertains.

As used herein when referring to numerical values or percentages, the term “about” includes variations due to the methods used to determine the values or percentages, statistical variance and human error. Moreover, each numerical parameter in this application should at least be construed in light of the number of reported significant digits and by applying ordinary rounding techniques.

The present disclosure relates to the targeted modification of the Amyloid β Precursor Protein (APP) gene, in embodiments modification of a wild type APP gene or an APP gene containing a Familial Alzheimer Disease (FAD) mutation, to introduce one or more protective mutations that may protect against Alzheimer disease (AD) and/or age-related cognitive decline or impairment. In embodiments, one to three protective mutations may be introduced. In embodiments, such protective mutations include: the Icelandic mutation (A673T)^{11,12,} the A673V^{13, 14} mutation and the H684R¹⁵ mutation. The A673T mutation reduces the secretion and accumulation of the toxic amyloid β peptide (Aβ1-42) in human cells^{11, 12}. The H684R¹⁵ facilitate the cleavage of the APP protein by BACE1 in the β′ position thus increasing the formation of Aβ11-xx peptides, which are non-amyloidogenic. The A673V^{13, 14} mutation also prevents AD only in heterozygote carriers by making the amyloid bodies less stable.

In embodiments, the present disclosure relates to modification of exon 16 of an APP gene at one or more nucleotide pairs, to convert C/G to T/A and/or A/T to G/C. In embodiments, such modifications result in an Alanine to Threonine or Valine change at position 673 and/or a Histidine to Arginine change at position 684. The latter modification of a Histidine in position 684 into an Arginine was also done in APP mRNA.

In an aspect, the present disclosure relates to introducing one or more modifications into the APP gene or mRNA by base or prime editing using site-specific cytidine or adenosine deaminases. The modifications introduced are designed to decrease the production of amyloidogenic Aβ peptides (e.g., by reducing APP protein processing by BACE1, by favoring the formation of non-amyloidogenic peptides or decreasing the stability of the amyloid bodies). In an aspect, a guide RNA may be designed and used in combination with a Cas9 nickase fused with a cytidine deaminase or a reverse transcriptase enzyme to specifically modify a cytidine (C) into a thymidine (T) in the targeted APP gene. In another aspect of the invention, a guide RNA may be designed and used in combination with a Cas9 nickase fused with an adenosine deaminase or a reverse transcriptase enzyme to specifically modify an adenosine (A) into an inosine (I), which is similar to a guanine (G) in the targeted APP gene. The latter modification may also be made in the APP mRNA with the REPAIRv2¹⁶ or the ADAR2 enzyme¹⁷. The target gene or mRNA thus modified, will ultimately reduce the level or the aggregation of amyloidogenic Aβ peptides produced by the cell. The present disclosure further relates to uses of such guide RNAs, site-specific cytidine or adenosine deaminase or REPAIRv2 or ADAR2 for decreasing Aβ-peptide expression/levels in a cell. The present disclosure also concerns uses of such guide RNAs, site-specific cytidine or adenosine deaminases, REPAIRv2 and ADAR2 for the prevention or treatment of Alzheimer's disease and/or age-related cognitive decline.

Accordingly, in an aspect, the present disclosure provides a method for decreasing APP protein processing into Aβ peptide by a cell comprising introducing at least one modification in the cell genome or in the cell mRNAs, wherein the modification decreases the amount or the aggregation of amyloidogenic Aβ peptides produced by the cell.

In an embodiment, the target gene encodes the APP protein.

Mutations of APP that are Responsible for Early Onset Familial Alzheimer's Disease

Over thirty coding mutations in the APP gene have been identified⁶. Twenty-five of these mutations are pathogenic, usually resulting in early onset autosomal dominant familial Alzheimer's disease (FAD) (FIG. 2). Substitutions at or near the β- and γ-proteolytic sites result in over-production of either total Aβ or a shift in the Aβ1-40:Aβ1-42 ratio towards formation of the more amyloidogenic Aβ1-42 peptide. On the other hand, substitutions within the amyloid-β peptides result in formation of Aβ peptides, which aggregate more easily¹⁸. Mutations in APP gene are also responsible for the common late-onset form of AD. Each of these pathogenic mutations may be corrected by base or prime editing.

Mutations that are Protective Against Alzheimer's Disease (AD): A673T, A673V and H684R

A673T mutation: Jonsson et al.¹² searched for low-frequency variants in the APP gene, which significantly reduce the risk of Alzheimer's disease. They studied coding variants in APP in whole-genome sequence data obtained from 1,795 Icelanders. They reported a coding mutation, i.e., an alanine to threonine substitution at position 673 in the APP gene (A673T), which protects against AD. This mutation is adjacent to the aspartyl protease β-site in APP and is located at position 2 in the Aβ peptide, and the proximity of A673T mutation to the proteolytic site of BACE1 may suggest that this variant may result in impaired BACE1 cleavage of APP. The A673T mutation reduces by about 40% the formation of Aβ peptides in vitro¹². The strong protective effect of the A673T mutation against AD provided a proof of principle that reducing the β-cleavage of APP may protect against the disease. Moreover, the A673T mutation also protects against cognitive decline in the elderly without a familial form of AD. The carriers of the A673T mutation have a 1.47 times greater chance of reaching the age of 85 than non-carriers. Jonsson et al.¹² concluded that the A673T mutation confers a strong protection against AD. Kero et al.¹¹ found the A673T variant in one person who died at the age of 104.8 years with little β-amyloid pathology. This observation supports the concept that this variant protects the brain against β-amyloid pathology and AD.

A673V mutation: Di Fede et al.¹⁹ found that the A673V mutation causes a FAD only in the homozygous state consistent with a recessive inheritance. The A673V APP mutation enhances β-amyloid (Aβ) production and markedly increases the aggregation and fibrilogenic properties of both Aβ1-40 and Aβ1-42^{14, 19, 20}. However, co-incubation of mutated and wild-type peptides confers instability to the Aβ aggregates and thus inhibits amyloidogenesis and neurotoxicity^{13, 21, 22.} The highly amyloidogenic effect of the A673V mutation in the homozygous state and its anti-amyloidogenic effect in the heterozygous state account for the autosomal recessive pattern of inheritance. Cimini et al.²² fused the first six residues of the Aβ1-42A2V to TAT (Aβ1-6A2VTAT(D)) to generate a cell permeable peptide, which conferred in vitro and in vivo neuroprotection against synaptopathy.

H684R mutation: The β′ site cleavage of mouse APP is more common than the corresponding cleavage of human APP. Thus normal mice do not accumulate β amyloid plaques and to not develop AD. There are three amino acids that differ between the human and the mouse APP protein near the β and β′ sites (i.e., Arg at position 676 in human APP (hAPP) is Gly (R676G) in mouse APP, Tyr681 is Phe (Y681F), and His at position 684 is Arg (H684R). Kimura et al.¹⁵ found that the H684R substitution within human APP protein, facilitated cleavage at the β′ site irrespective of the species origin of BACE1, thereby significantly increasing the level of amyloidolytic Aβ11-XX and decreasing the level of the amyloidogenic Aβ1-XX.

In an embodiment, the methods described herein comprise providing the cell with a site-specific cytidine deaminase, adenosine deaminase or Cas-nickase-reverse transcriptase specifically targeting a nucleic acid sequence in the endogenous APP polynucleotide gene or mRNA sequence of the cell.

In an embodiment, the site-specific endonuclease is a Cas9 nickase fused with a cytidine deaminase (e.g., APOBEC1 (apolipoprotein B editing complex 1) or Target-AID) or an adenosine deaminase such as ABE7.10²³, REPAIRv2¹⁶ or ADAR2¹⁷ or a reverse transcriptase²⁴.

In an embodiment, the at least one modification induced by the cytidine or adenosine deaminase or reverse transcriptase results in an APP polynucleotide gene sequence encoding an APP protein in which the alanine at position 673 and/or the histidine in position 684 has been substituted by another amino acid. In an embodiment, the at least one modification changes the alanine at position 673 of the APP protein into a threonine or a valine, and/or changes the histidine in position 684 into an arginine. In another embodiment, the at least one modification induced by the cytidine or adenosine deaminase complex, the REPAIRv2 or the ADAR2 results in a modification of one or more amino acids recognized by an α, β, or γ secretase in the APP protein encoded by the APP polynucleotide gene sequence. In an embodiment, the at least one modification reduces the risk of developing Alzheimer's disease.

In an embodiment, the site-specific cytidine or adenosine deaminase is a Cas nickase (e.g., derived from either SpCas9, SaCas9, CjCas9, ScCas9, CasX, CasY, Cpf1, eSpCas9, eSaCas9, HiFi Cas9™ (IDT Inc.)), fused to an ADAR2 deaminase or a REPAIRv2 deaminase and the method further comprises providing the cell with at least on one guide RNA comprising a nucleotide seed region complementary to a target sequence in the endogenous APP polynucleotide gene or mRNA sequence of the cell.

In an embodiment, the guide RNA comprises a nucleotide seed region of at least 8 nucleotides perfectly complementary to the target nucleic acid sequence in the endogenous APP polynucleotide gene, wherein the target nucleic acid sequence is immediately adjacent to a protospacer adjacent motif (PAM) recognized by a Cas ribonucleoprotein complex comprising the Cas9 nickase derived from a Cas9 nuclease (e.g., SpCas9, SaCas9, ScCas9, CasX, CasY, Cpf1, eSpCas9, eSaCas9, HiFi Cas9™). The sequences coding for some of these enzymes have been optimized for humans: hSpCas9, hSaCas9, hCjCas9.

In embodiments, the PAM comprises a AAA, AAG, ACA, ATG, CC, CM, CCN, GM, GAT, GGA, GGC, GGT, GTA, GTC, GTT, TCN, NG, NGG, NNG, NAG, NGA, NNAGAA, NNGAAT, NNGAGT, NNGGAT, NNGGGT, NNNAAT NNNAGT, NNNGAT, NNNGCA, NNNGGT, NNNNACA, NNNNACAC, NNNNATAC, NNNNGAAT, NNNNGATT, NNNNGCAC, NNNNGTAC, NNNNGTAT, NNNNGTTT, TGA, TGC, TGT, TTA, TTC, TTT, TTTA or a TTTC sequence.

In an embodiment, the target nucleic acid sequence of the above-mentioned guide RNA is located in the APP polynucleotide gene sequence.

In an embodiment, the above-mentioned target nucleic acid sequence of the guide RNA comprises the following nucleic acid sequence of the human APP polynucleotide gene sequence having reference number NCBI NG_007376.1. (The PAM sequence is in bold)

For the A673T mutation:
(SEQ ID NO: 53)
ATTCTGCATCCATCTTCACTTCAGAG
(SEQ ID NO: 54)
TTCTGCATCCATCTTCACTTCAGAG
(SEQ ID NO: 55)
TCTGCATCCATCTTCACTTCAGAG
(SEQ ID NO: 56)
CTGCATCCATCTTCACTTCAGAG
(SEQ ID NO: 57)
TGCATCCATCTTCACTTCAGAG
(SEQ ID NO: 58)
GCATCCATCTTCACTTCAGAG
(SEQ ID NO: 59)
ATTCTGCATCCATCTTCACTTCAGAGAT
(SEQ ID NO: 60)
TTCTGCATCCATCTTCACTTCAGAGAT
(SEQ ID NO: 61)
TCTGCATCCATCTTCACTTCAGAGAT
(SEQ ID NO: 62)
CTGCATCCATCTTCACTTCAGAGAT
(SEQ ID NO: 63)
TGCATCCATCTTCACTTCAGAGAT
(SEQ ID NO: 64)
GCATCCATCTTCACTTCAGAGAT
(SEQ ID NO: 65)
ATTCTGCATCCAGATTCACTTCAGAG
(SEQ ID NO: 66)
TTCTGCATCCAGATTCACTTCAGAG
(SEQ ID NO: 67)
TCTGCATCCAGATTCACTTCAGAG
(SEQ ID NO: 68)
CTGCATCCAGATTCACTTCAGAG
(SEQ ID NO: 69)
TGCATCCAGATTCACTTCAGAG
(SEQ ID NO: 70)
GCATCCAGATTCACTTCAGAG
(SEQ ID NO: 71)
ATTCTGCATCCAGATTCACTTCAGAGAT
(SEQ ID NO: 72)
TTCTGCATCCAGATTCACTTCAGAGAT
(SEQ ID NO: 73)
TCTGCATCCAGATTCACTTCAGAGAT
(SEQ ID NO: 74)
CTGCATCCAGATTCACTTCAGAGAT
(SEQ ID NO: 75)
TGCATCCAGATTCACTTCAGAGAT
(SEQ ID NO: 76)
GCATCCAGATTCACTTCAGAGAT
For the A673V mutation:
(SEQ ID NO: 77)
ATGGATGCAGAATTCCGACATGAC
(SEQ ID NO: 78)
TGGATGCAGAATTCCGACATGAC
(SEQ ID NO: 79)
GGATGCAGAATTCCGACATGAC
(SEQ ID NO: 80)
GATGCAGAATTCCGACATGAC
(SEQ ID NO: 81)
ATGCAGAATTCCGACATGAC
(SEQ ID NO: 82)
TGCAGAATTCCGACATGAC
For the H684R mutation:
(SEQ ID NO: 83)
TGAAGTTCATCATCAAAAATTGG
(SEQ ID NO: 84)
GAAGTTCATCATCAAAAATTGG
(SEQ ID NO: 85)
AAGTTCATCATCAAAAATTGG
(SEQ ID NO: 86)
AGTTCATCATCAAAAATTGG
(SEQ ID NO: 87)
GTTCATCATCAAAAATTGG
(SEQ ID NO: 88)
TTCATCATCAAAAATTGG
(SEQ ID NO: 89)
CATCATCAAAAATTGGTACGTAAAAT
(SEQ ID NO: 90)
ATCATCAAAAATTGGTACGTAAAAT
(SEQ ID NO: 91)
TCATCAAAAATTGGTACGTAAAAT
(SEQ ID NO: 92)
CATCAAAAATTGGTACGTAAAAT
(SEQ ID NO: 93)
ATCAAAAATTGGTACGTAAAAT
(SEQ ID NO: 94)
TCAAAAATTGGTACGTAAAAT

In an embodiment, the at least one modification within the endogenous APP polynucleotide gene sequence of the cell is introduced by base or prime editing.

In another aspect, the present disclosure provides a guide RNA as defined herein, which in embodiments can be used in the methods described herein. The present disclosure also provides a vector comprising a nucleic acid encoding a guide RNA or a pegRNA described herein. Also provided are cells comprising such a nucleic acid or vector. In an embodiment, the vector is a viral vector. In an embodiment, the viral vector is a lentiviral vector, Adeno-Associated viral (AAV) vector, adenovirus viral vector or herpes virus vector. In an embodiment, the vectors are virus like particles (VLPs), extra-cellular vesicles or exosomes.

In a particular embodiment, the above-mentioned vectors further comprise a nucleic acid encoding a site-specific deaminase or reverse transcriptase. In an embodiment, the vector further comprises a nucleic acid encoding a nickase Cas9 fused with a cytidine deaminase, an adenosine deaminase or a reverse transcriptase, a REPAIRv2 adenosine deaminase or ADAR2 adenosine deaminase.

In a further aspect, the present disclosure relates to a composition comprising: i) one or more of the above-mentioned guide RNAs or pegRNAs, ii) a cytidine deaminase, an adenosine deaminase or a reverse transcriptase, iii) one or more of the above-mentioned vectors, and a carrier, such as a biologically acceptable carrier or a pharmaceutically acceptable carrier.

In an embodiment, the composition further comprises a site-specific deaminase or reverse transcriptase or a nucleic acid encoding a site-specific deaminase or reverse transcriptase. In an embodiment, the site-specific deaminase is an inactive Cas9 nuclease or nickase fused with a cytidine deaminase, an adenosine deaminase or a reverse transcriptase.

In an embodiment, the present relates to the above-mentioned guide RNAs or pegRNAs, nucleic acids vectors, host cells and/or compositions for use in decreasing Amyloid Precursor Protein (APP) processing into amyloidogenic Aβ peptides by a cell, or for decreasing the production of amyloidogenic Aβ peptides by a cell.

In another embodiment, the present disclosure concerns the above-mentioned guide RNAs or pegRNAs, nucleic acids, vectors, host cells and/or compositions for use in treating or preventing Alzheimer's disease or age-related cognitive decline in a subject in need thereof.

In a related aspect, the present disclosure concerns the above-mentioned guide RNAs or pegRNAs, vectors, host cells and/or compositions for the preparation of a medicament for decreasing Amyloid Precursor Protein (APP) processing into amyloidogenic Aβ peptides by a cell.

The present disclosure also concerns the above-mentioned guide RNAs or pegRNAs, deaminases or reverse transcriptase, vectors, host cells and/or compositions for the preparation of a medicament for treating or preventing Alzheimer's disease or age-related cognitive decline in a subject in need thereof.

The present disclosure further concerns the use of the above-mentioned guide RNAs or pegRNAs, deaminases or reverse transcriptase, vectors, host cells and/or compositions for decreasing Amyloid β Precursor Protein (APP) processing into Aβ peptide by a cell.

In another embodiment, the present disclosure relates to the use of the above-mentioned guide RNAs or pegRNAs, deaminases or reverse transcriptase, vectors, host cells and/or compositions for preventing or treating Alzheimer's disease or age-related cognitive decline in a subject in need thereof.

In another embodiment, the present disclosure relates to a method of preventing or treating Alzheimer's disease or age-related cognitive decline in a subject in need thereof, comprising introducing into or contacting a cell of the subject with the above-mentioned guide RNAs or pegRNAs, deaminases or reverse transcriptase, vectors, host cells and/or compositions.

In an embodiment of the above-mentioned methods, the cell is from subject in need thereof. In a particular embodiment, the subject in need thereof is a subject at risk of developing Alzheimer's disease.

The present disclosure shows that it is possible to introduce one or more modifications into a target gene or mRNA involved in Aβ peptide production by gene editing using one or more site-specific deaminases or reverse transcriptase. The modifications introduced are specifically designed to decrease the production of amyloidogenic Aβ peptides.

Applicants show herein that the endogenous APP polynucleotide gene or mRNA sequence within a cell may be efficiently genetically modified to introduce one or more modifications designed to decrease amyloidogenic Aβ-peptide production. The modifications are introduced by providing the cell with a site-specific deaminase or reverse transcriptase specifically targeting the endogenous APP polynucleotide gene or mRNA sequence.

A deamination reaction is produced by the site-specific deaminase in the endogenous APP polynucleotide gene or mRNA sequence thereby modifying it.

Using such methods, modifications into the target gene or mRNA can be made to correct an endogenous mutation associated with increased risk of amyloidogenic Aβ peptide production or accumulation or to introduce one or more modifications which decrease APP expression or processing into amyloidogenic Aβ peptides or production of amyloidogenic Aβ peptides.

For example, mutations in the APP gene associated with familial forms of Alzheimer's disease may be corrected in accordance with the present disclosure by targeting the endogenous APP gene or mRNA with a deaminase or reverse transcriptase changing the mutated codon to the wild-type one. In another aspect of the present disclosure, the APP gene may be modified to introduce a protective mutation into the APP gene associated with decreased levels of amyloidogenic Aβ-peptide production or accumulation.

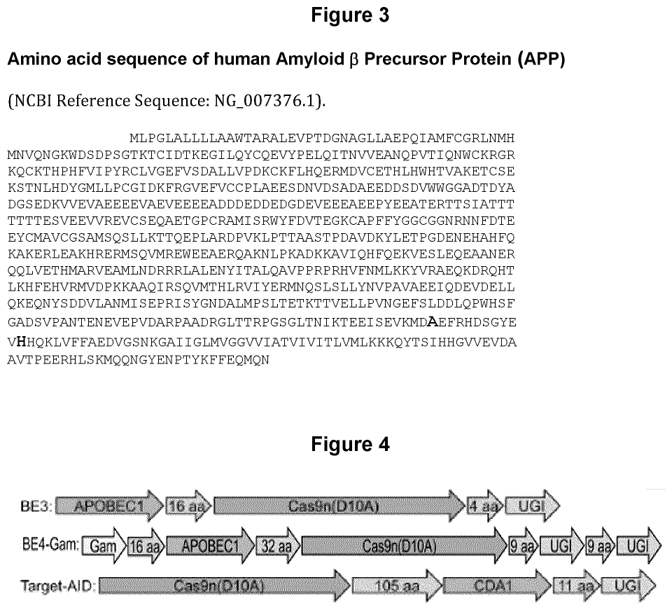

As used herein, the terms “APP gene”, “APP nucleic acid” and APP polynucleotide sequence” are used interchangeably and refer to the nucleic acid sequence encoding the Amyloid Precursor Protein (Entrez 351; Ensembl ENSG00000142192; UniProt P05067; RefSeq mRNA NM_000484; RefSeq (protein) NP_000475). A wild-type APP nucleic acid is a nucleic acid, which has the nucleotide sequence of the APP gene naturally found in subjects and which does not comprise mutations (in the coding region of the APP protein or elsewhere in the APP gene), which are associated with an increased risk of developing Alzheimer's disease. A wild type APP nucleic acid thus includes allelic variants not associated with familial forms of Alzheimer's disease and encodes the wild type APP protein (e.g., NM_000484.3; Uniprot P05067, and FIG. 3). In a particular embodiment, the wild type APP nucleic acid sequence has the sequence of NCBI Reference Sequence: NG_007376.1 (SEQ ID NO: 1; Exons: 4995 . . . 5251, 63670 . . . 63837, 85745 . . . 85874, 122469 . . . 122581, 124624 . . . 124817, 153775 . . . 153977, 175636 . . . 175803, 178402 . . . 178458, 193343 . . . 193476, 199792 . . . 199866, 200592 . . . 200750, 220064 . . . 220192, 221130 . . . 221229, 263859 . . . 264080, 270744 . . . 270797, 278148 . . . 278248, 283953 . . . 284099, 294051 . . . 295272). An endogenous APP polynucleotide gene sequence in a cell refers to the unmodified APP nucleic acid sequence found in such cell.

Many mutations causing Alzheimer's disease have been reported in the APP gene and these may be corrected in accordance with the present disclosure. In a particular aspect of the present disclosure, the correction involves the replacement of the mutated nucleotide(s) with nucleotide(s) normally found in the APP gene. In another aspect, the correction involves the replacement of the mutated nucleotides with nucleotides encoding the wild type amino acids of the APP protein. In such a case, the replacement nucleotides may be the same as those found in the wild type APP nucleic acid sequence or may be different (e.g., due to codon degeneracy), as long as the corrected sequence encodes the wild type APP protein. Mutations in the exons encoding the APP protein, in the promoter or in any other regulatory sequence in the APP gene modulating the expression of the APP protein may be targeted in accordance with the present disclosure. For example, mutations in any of the 18 exons of the APP gene may be targeted and corrected in accordance with the present disclosure (i.e., mutations in exon 1, exon 2, exon 3, exon 4, exon 5, exon 6, exon 7, exon 8, exon 9, exon 10, exon 11, exon 12, exon 13, exon 14, exon 15, exon 16, exon 17 and exon 18). Modifications introduced in the portion of the polynucleotide gene sequence encoding the APP protein (e.g., exon sequences) will be reflected in the APP protein expressed by the cell (i.e., the cell will normally express a modified/corrected APP protein).

Non-limiting examples of mutations in the APP protein that may be corrected include: KM670/671NL (12), Ala673Val (13), His677Arg (14), Asp678Asn (15), Asp678His (16), Glu682Lys (17), Ala692Gly (18), Glu693Gln (19), Glu693Gly (20), Glu693del (21), Asp694Asn (22), Leu705Val (23), Ala713Thr (24), Ala713Thr (25), Ala713Val (26), Thr714Ala (27), Thr714Ile (28), Val715Met (29), Ile716Val (30), Ile716Phe (31), Val717Ile (32), Val717Leu (33), Val717Phe (34), Val717Gly (35), Leu723Pro (36), Lys724Asn (37) and His733Pro (38). All mutations located between amino acids 656 and 688 are located in exon 16 of the APP gene. This exon has been shown to be efficiently targeted and replaced using an embodiment of the method of the present disclosure.

Other mutations associated with Alzheimer's disease, such as those found in the Presenilin 1 (PS1) and Presenilin 2 (PS2) genes may also be corrected using the methods described herein.

In addition to correcting mutations associated with increased risk of developing Alzheimer's disease (e.g., mutations associated with familial forms of AD), it is also possible to introduce one or more modifications into the APP gene, which are known to protect against Alzheimer's disease. One such modification is the replacement of the alanine at position 673 of the APP protein sequence with a threonine (A673T substitution in exon 16). The presence of the A673T variant in subjects has been found to reduce the risk of developing Alzheimer's disease and to protect against age-related cognitive decline. The A673T substitution is adjacent to the β-secretase cleavage site in APP and results in a 40% reduction in the formation of toxic amyloid-6 peptides in 293T cells. A second modification is the replacement of the alanine at position 673 of the APP protein sequence with a valine (A673V substitution in exon 16). Although the A673V mutation induces Alzheimer disease when the patient is homozygous for the mutation, this mutation in heterozygous humans is protective against AD^{13-15, 22}. A third modification is the replacement of the histidine at position 684 of the APP protein sequence with an arginine (H684R substitution in exon 16). BACE1 normally cuts in human APP protein between amino acid methionine at position 671 and asparagine at position 672, the H684R mutation favors the cutting by between amino acid tyrosine at position 681 and glutamic acid at position 682 thus leading to the formation of shorter A1111-xx peptides which are not amyloidogenic¹⁵.

Introduction of the A673T, A673V or H684H substitution in accordance with the present disclosure may be made alone in the APP gene, or in combination with one or more other modifications aiming, for example, either at correcting endogenous mutations associated with an increased risk of developing AD or age-related cognitive decline or at introducing mutations, which are preventing AD. For example, mutations associated with increased risk of developing AD present in exon 16 may advantageously be corrected at the same time as the A673T, A673V or H684H protective substitution is introduced in the APP gene. Of course, mutations located elsewhere (i.e., in exons other than exon 16) may also be concurrently corrected.

Modifications in the APP gene or other genes associated with AD may be made in cells (neurons) of a subject in need thereof. As used herein, “a subject in need thereof” is a subject, which may benefit from a decreased production of amyloidogenic Aβ peptides. Non-limiting examples of a subject in need thereof include a subject having cells showing an increased level of amyloidogenic Aβ peptide production (APP expression and/or APP maturation) or activity as compared to cells from a normal subject. In an embodiment, the subject in need thereof is a healthy subject (e.g., a subject at risk of developing AD or age-related cognitive decline) a subject already diagnosed with AD or age-related cognitive decline or a subject who has not been diagnosed with AD and who does not have a mutation known to increase the risk of AD. As used herein, a subject at risk of developing AD or age-related cognitive decline, is a subject that has not yet been diagnosed with the disease or condition but which, due to certain factors (age, familial history, heredity) is likely to develop de disease or condition later on in his/her life. In an embodiment, the subject at risk is a subject having a mutation in the APP, PS-1 and/or PS-2 gene(s). In another embodiment, the subject at risk is a subject having a mutation in the APOE-e4 gene. In another embodiment, the subject at risk is a subject having at least one family member (e.g., a mother, father, brother, sister or child) diagnosed with Alzheimer's disease or age-related cognitive decline. In an embodiment, the subject at risk is a subject having a mutation associated with early onset Alzheimer's disease or familial Alzheimer's disease (FAD). In an embodiment, the subject is a mammal, preferably, a human.

Modifications in genes associated with AD (e.g., APP, PS-1 and PS-2) in accordance with the present disclosure can be used to prevent or treat Alzheimer's disease or age-related cognitive decline. As used herein, the term “prevention/preventing/prevent” means that the modification(s) avoid(s) or delay(s) the onset of the disease. As used herein, the term “treat/treating/treatment” includes instances where the genetic modification(s) reduce(s) partially or completely the progression of the disease and instances where symptoms associated with the disease are reduced partially or completely (i.e., one or more symptoms associated with Aβ peptide neurotoxicity).

Preferably, the one or more modifications in a targeted gene (e.g., APP) in cells (e.g., neurons) of a subject are introduced as early as possible after the identification of a risk of developing AD or soon after AD diagnosis. In a particular embodiment, the one or more genetic modifications in cells are made after the detection by Magnetic Resonance Imaging (MRI) of plaques comprising extracellular deposits of amyloid β (Aβ) peptides in the subject's brain.

Methods of introducing one or more genetic modifications in a targeted gene in accordance with the present disclosure preferably involve Base editing (BE) or prime editing (PE). The proposed treatment requires the modification of base pairs in the targeted gene (e.g., APP) using specifically designed Cas nickase fused with a deaminase or a reverse transcriptase, such as the REPAIRv2 deaminase or the ADAR2 deaminase. The introduction of a SSB enable to modify the targeted gene to, for example, reduce the formation of toxic Aβ peptides (e.g., by correcting one or more endogenous mutations in a targeted gene, which are associated with increased risk to develop AD and/or by introducing a protective modification in the targeted gene, which reduces Aβ plaque formation in neurons).

Although genetic modifications in a targeted gene are preferably introduced by Base or prime editing HR, SSBs in cells, DNA might also be spontaneously repaired by Non-Homologous End Joining (NHEJ) leading to the presence micro-insertions or micro-deletions (INDELs) in the targeted gene. Although these forms of genetic modifications are not preferred they can nevertheless be useful to prevent the formation of the Aβ peptide (e.g., by for example reducing completely or partially the level of APP synthesized in cells or by preventing a cut of the APP protein by the β-secretase.

Various types of endonucleases or nickases may be fused with a deaminase or reverse transcriptase to induce a site-specific base mutation at selected site(s) in a targeted gene(s) or mRNA(s) (e.g., APP, PS-1, PS-2, etc.). Non-limiting examples of useful endonucleases and nickases include meganucleases, Zinc Finger Nucleases (ZFNs), Transcription Activator-Like Effector nucleases (TALENs), the Cas nucleases, Cas nickases or an inactive Cas nuclease used with a guide RNA in the Clustered regularly interspaced short palindrome repeat (CRISPR) system²⁵⁻²⁷. Each of these technologies can be used to modify a targeted gene in accordance with the present disclosure.

Preferably, the present disclosure uses the CRISPR system (i.e., combination of guide RNA or pegRNA and Cas nickase fused with a deaminase or a reverse transcriptase, the REPAIRv2 deaminase or the ADAR2 deaminase) to introduce one or more genetic modifications in a targeted gene or mRNA (e.g., APP). Applicants demonstrate herein that specific guide RNAs or pegRNA can be rapidly produced and used with a deaminase or reverse transcriptase to efficiently modify the endogenous APP gene in human cells.

### The Crispr System

Recent discoveries in the field of bacterial immunity have led to the development of a new system for controlling gene expression in cells. Bacterial and archaea have developed adaptive immune defenses termed clustered regularly interspaced short palindromic repeats (CRISPR) systems, which use crRNAs and Cas proteins to degrade complementary sequences present in invading viral and plasmid DNA²⁸. Jinek et al.²⁹ and Mali et al.²⁸ have engineered a type II bacterial CRISPR system using custom guide RNA to induce a double strand break (DSB) in DNA. This permits to modify a gene targeted by the guide RNA by homology directed repair (HDR), which requires the presence of a donor DNA containing homology sequences that precede and follow the DSB³⁰.

Subsequently the CRISPR/Cas technology has been improved to permit base editing without inducing a DSB. The first method, called base editing, uses a guide RNA and a Cas9 nickase (cutting only one DNA strand) fused with a cytidine deaminase to chemically modify a cytidine into a thymine³¹. The second base editing method uses a guide RNA and a Cas9 nickase fused with an adenosine deaminase to chemically modify an adenosine into an inosine, the equivalent of a guanine²³. The third method, called PRIME editing, uses an extended guide RNA (called a pegRNA) and a Cas9 nickase fused with a reverse transcriptase (the fusion protein is e.g., PE2) to replace any nucleotide by any other nucleotide²⁴. It is described herein that the Cas9 nickase fused either with a deaminase or reverse transcriptase to efficiently modify a targeted gene (e.g., APP) in cells. Accordingly, a protective modification in the APP gene (i.e., either the A673T, A673V or H684H mutation in Exon 16 of the APP gene) was introduced. This modification has been shown to reduce amyloidogenic Aβ peptide formation in cells.

Accordingly, in an aspect, methods of the present disclosure involve the design of one or more guide RNAs for inducing a base pair modification in the APP gene or a nucleotide in the mRNA. The guide RNA(s) or pegRNA targeting a region of interest and Cas nickase (e.g., Cas9n) fused with a deaminase or reverse transcriptase, the REPAIRV2 deaminase or ADAR2 deaminase are then used to introduce the desired modification(s) in the endogenous gene within the cell. The present disclosure further relates to uses of such targeted genetic modification(s), such as for reducing amyloidogenic Aβ peptide formation in cells of a subject in need thereof, such as for the treatment of Alzheimer's disease and/or age-related cognitive decline.

In order to induce a base pair modification in the DNA at a specific site, the Cas9-deaminase or reverse transcriptase protein requires the presence of a guide RNA or pegRNA and a protospacer adjacent motif (PAM) immediately following the nucleotide sequence targeted by the guide RNA³². The PAM is the sequence of nucleotides adjacent to the nucleotide sequence targeted by the guide RNA. Different Cas proteins require a different PAM. Accordingly, selection of a specific polynucleotide target sequence (e.g., on the APP nucleic acid sequence) by a guide RNA is generally based on the recombinant Cas protein used.

In embodiments, the recombinant Cas protein that may be used in accordance with the present disclosure is i) derived from a naturally occurring Cas; and ii) has a nickase activity to introduce a single strand break (SSB) in cellular DNA when in presence of appropriate guide RNA(s). In another embodiment, the Cas protein is a Cas9 protein having a nickase activity [39]. In accordance with the present disclosure, the Cas protein can be derived from any naturally occurring source. In another embodiment, the adenosine may be modified into an inosine which is biochemically read as guanosine by targeting the gene or the mRNA with a guide RNA and with an adenosine deaminase such as ADAR2 or REPAIRv2.

For example, Cas9 proteins are natural effector proteins produced by numerous species of bacteria including Streptococcus pyogenes³³, Streptococcus thermophilus³⁴ Campylobacter jejuni³⁵, Staphylococcus aureus³⁶, Streptococcus canis³⁷ and Neisseria meningitides³² Accordingly, in an embodiment, the Cas protein of the present disclosure is a Cas9 nickase derived from the above mentioned bacteria. In an embodiment, the Cas9 recombinant protein of the present disclosure is a human-codon optimized spCas9 derived from the above-mentioned bacteria.

Although a perfect match between the guide RNA or pegRNA and the DNA sequence on the targeted gene is preferred, a mismatch between a guide RNA or pegRNA and a target base-pair on the gene sequence of interest is also permitted as along as it still allows an hybridization of the guide RNA or pegRNA with the targeted DNA sequence followed by a Cas9 recognition and introduction of a nucleotide modification in the targeted DNA or mRNA. A seed sequence of between 8-12 nucleotides on the guide RNA or pegRNA perfectly complementary to the target sequence (e.g., APP gene or mRNA sequence) is preferred for proper recognition of the target sequence. In general, guide RNA or pegRNA activity is inversely correlated with the number of mismatches. Preferably, the guide RNA or pegRNA of the present disclosure comprises 7 mismatches, 6 mismatches, 5 mismatches, 4 mismatches, 3 mismatches, more preferably 2 mismatches, or less, and even more preferably no mismatch, with the corresponding target gene sequence. Of course, the smaller the number of nucleotides in the guide RNA or pegRNA the smaller the number of mismatches tolerated. The binding affinity is thought to depend on the sum of matching guide RNA-DNA, pegRNA-DNA or guide RNA-mRNA combinations.

In addition to deaminases fused with Cas9 derived nickases, other deaminases may be used in accordance with the present disclosure to introduce site-specific nucleotide modification into DNA or mRNA. Such deaminase may be fused with Cas9 nickases but may also be fused with meganucleases, Zinc finger nucleases and transcription activator-like effector nucleases (TALENs).

The Cas nickase recombinant proteins of the present disclosure preferably comprise at least one Nuclear Localization Signal (NLS) to target the protein into the cell nucleus. Accordingly, as used herein the expression “nuclear localization signal” or “NLS” refers to an amino acid sequence, which ‘tags’ a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal, which targets proteins out of the nucleus. Classical NLSs can be further classified as either monopartite or bipartite. The first NLS to be discovered was the sequence PKKKRKV (SEQ ID NO: 95) in the SV40 Large T-antigen (a monopartite NLS). The NLS of nucleoplasmin, KR[PAATKKAGQA]KKKK (SEQ ID NO: 96), is the prototype of the ubiquitous bipartite signal: two clusters of basic amino acids, separated by a spacer of about 10 amino acids.

There are many other types of NLS, which are qualified as “non-classical”, such as the acidic M9 domain of hnRNP A1, the sequence KIPIK in yeast transcription repressor Mata2, the complex signals of U snRNPs as well as a recently identified class of NLSs known as PY-NLSs. Thus, any type of NLS (classical or non-classical) may be used in accordance with the present disclosure as long as it targets the protein of interest into the nucleus of a target cell. In an embodiment, the NLS is derived from the simian virus 40 large T antigen. In an embodiment, the NLS of the recombinant protein of the present disclosure comprises the following amino acid sequence: SPKKKRKVEAS (SEQ ID NO: 97). In an embodiment the NLS comprises the sequence KKKRKV (SEQ ID NO: 98). In an embodiment, the NLS comprises the sequence SPKKKRKVEASPKKKRKV (SEQ ID NO: 99). In another embodiment, the NLS comprises the sequence KKKRK (SEQ ID NO: 100).

The Cas nickase protein of the present disclosure may optionally advantageously be coupled to a protein transduction domain to ensure entry of the protein into the target cells. Alternatively, the nucleic acid coding for the guide RNA or pegRNA and for the deaminase or reverse transcriptase may be delivered in targeted cells using various viral vectors, virus like particles (VLP) or exosomes.

Protein transduction domains (PTD) may be of various origins and allow intracellular delivery of a given therapeutic by facilitating the translocation of the protein/polypeptide into a cell membrane, organelle membrane, or vesicle membrane. PTD refers to a polypeptide, polynucleotide, carbohydrate, or organic or inorganic compound that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle including the mitochondria.

In an embodiment, a PTD is covalently linked to the amino terminus of a recombinant protein of the present disclosure. In another embodiment, a PTD is covalently linked to the carboxyl terminus of a recombinant protein of the present disclosure. Exemplary protein transduction domains include but are not limited to a minimal undecapeptide protein transduction domain (corresponding to residues 47-57 of HIV-1 TAT comprising YGRKKRRQRRR; a polyarginine sequence comprising a number of arginines sufficient to direct entry into a cell (e.g., 3, 4, 5, 6, 7, 8, 9, 10, or 10-50 arginines); a VP22 domain (49); an Drosophila Antennapedia protein transduction domain (50); a truncated human calcitonin peptide (51); RRQRRTSKLMKR (SEQ ID NO: 101); Transportan GWTLNSAGYLLGKINLKALAALAKKIL (SEQ ID NO: 102); KALAWEAKLAKALAKALAKHLAKALAKALKCEA (SEQ ID NO: 103); and RQIKIWFQNRRMKWKK (SEQ ID NO: 104). Further exemplary PTDs include but are not limited to, KKRRQRRR (SEQ ID NO: 105), RKKRRQRRR (SEQ ID NO: 106); or an arginine homopolymer of from 3 arginine residues to 50 arginine residues.

Other non-limiting examples of PTD include an endosomal escape peptide. Non-limiting examples of such endosomal escape peptides are listed in the Table 1 below.

TABLE 1
Endosomal escape peptides

| | Primary | | | SEQ |
|---|---|---|---|---|
| | se- | | Refer- | ID |
| Peptide | quence | Mechanism | ences | NO: |
| DT | VGSS | Fusion | (52) | 107 |
| | LSCI | | | |
| | NLDW | | | |
| | DVIR | | | |
| | DKTK | | | |
| | TKIE | | | |
| | SLKE | | | |
| | HGPI | | | |
| | KNKM | | | |
| | SESP | | | |
| | NKTV | | | |
| | SEEK | | | |
| | AKQY | | | |
| | LEEF | | | |
| | HQTA | | | |
| | LEHP | | | |
| | ELSE | | | |
| | LKTV | | | |
| | TGTN | | | |
| | PVFA | | | |
| | GANY | | | |
| | AAWA | | | |
| | VNVA | | | |
| | QVID | | | |
| | SETA | | | |
| | DNLE | | | |
| | KTTA | | | |
| | ALSI | | | |
| | LPGI | | | |
| | GSVM | | | |
| | GIAD | | | |
| | GAVH | | | |
| | HNTE | | | |
| | EIVA | | | |
| | QSIA | | | |
| | LSSL | | | |
| | MVAQ | | | |
| | AIPL | | | |
| | VGEL | | | |
| | VDIG | | | |
| | FAAY | | | |
| | NFVE | | | |
| | SIIN | | | |
| | LFQV | | | |
| | VHNS | | | |
| | YNRP | | | |
| | AYSP | | | |
| | G | | | |
| GALA | WEAA | Membrane | (53) | 108 |
| | LAEA | destabilization, | | |
| | LAEA | pore | | |
| | LAEH | formation | | |
| | LAEA | and | | |
| | LAEA | flip-flop | | |
| | LEAL | of membrane | | |
| | AA | lipids | | |
| PEA | VLAG | Pore formation | (54) | 109 |
| | NPAK | | | |
| | FIDL | | | |
| | DIKP | | | |
| | TVIS | | | |
| | HRLH | | | |
| | FPEG | | | |
| | GSLA | | | |
| | ALTA | | | |
| | HQAC | | | |
| | HLPL | | | |
| | ETFT | | | |
| | RHRQ | | | |
| | PRGW | | | |
| | EQLE | | | |
| | QCGY | | | |
| | PVQR | | | |
| | LVAL | | | |
| | YLAA | | | |
| | RLSW | | | |
| | NQVD | | | |
| | QVIR | | | |
| | NALA | | | |
| | SPGS | | | |
| | GGDL | | | |
| | GEAI | | | |
| | REQP | | | |
| | EQAR | | | |
| | LALT | | | |
| INF-7 | GLFE | Membrane fusion | (55) | 110 |
| | AIEG | and | | |
| | FIEN | destabilization | | |
| | GWEG | | | |
| | MIDG | | | |
| | WYGC | | | |
| LAH4 | KKAL | Membrane | (56) | 111 |
| | LALA | destabilization | | |
| | LHHL | | | |
| | AHLA | | | |
| | LHLA | | | |
| | LALK | | | |
| | KA | | | |
| CM18 | KWKL | Membrane | (57) | 112 |
| | FKKI | destabilization | | |
| | GAVL | | | |
| | KVLT | | | |
| | TG | | | |
| HGP | LLGR | Pore | (58) | 113 |
| | RGWE | formation | | |
| | VLKY | and fusion | | |
| | WWNL | | | |
| | LQ | | | |
| | YWSQ | | | |
| | EL | | | |
| H5WYG | GLFH | Membrane | (59) | 114 |
| | AIAH | destabilization | | |
| | FIHG | | | |
| | GWHG | | | |
| | LIHG | | | |
| | WYG | | | |
| HA2 | GLFG | Membrane | (60) | 115 |
| | AIAG | Fusion | | |
| | FIEN | and | | |
| | GWEG | destabilization | | |
| | MIDG | | | |
| | WYG | | | |
| EB1 | LIRL | Membrane | (61) | 116 |
| | WSHL | destabilization | | |
| | IHIW | | | |
| | FQNR | | | |
| | RLK | | | |
| | WKKK | | | |
| DT | VGSS | Fusion | (52) | 117 |
| | LSCI | | | |
| | NLDW | | | |
| | DVIR | | | |
| | DKTK | | | |
| | TKIE | | | |
| | SLKE | | | |
| | HGPI | | | |
| | KNKM | | | |
| | SESP | | | |
| | NKTV | | | |
| | SEEK | | | |
| | AKQY | | | |
| | LEEF | | | |
| | HQTA | | | |
| | LEHP | | | |
| | ELSE | | | |
| | LKTV | | | |
| | TGTN | | | |
| | PVFA | | | |
| | GANY | | | |
| | AAWA | | | |
| | VNVA | | | |
| | QVID | | | |
| | SETA | | | |
| | DNLE | | | |
| | KTTA | | | |
| | ALSI | | | |
| | LPGI | | | |
| | GSVM | | | |
| | GIAD | | | |
| | GAVH | | | |
| | HNTE | | | |
| | EIVA | | | |
| | QSIA | | | |
| | LSSL | | | |
| | MVAQ | | | |
| | AIPL | | | |
| | VGEL | | | |
| | VDIG | | | |
| | FAAY | | | |
| | NFVE | | | |
| | SIIN | | | |
| | LFQV | | | |
| | VHNS | | | |
| | YNRP | | | |
| | AYSP | | | |
| | G | | | |

In an embodiment, the protein transduction domain is TAT or Pep-1. In an embodiment, the protein transduction domain is TAT and comprises the sequence SGYGRKKRRQRRRC (SEQ ID NO: 118). In another embodiment, the protein transduction domain is TAT and comprises the sequence YGRKKRRQRRR (SEQ ID NO: 119). In another embodiment, the protein transduction domain is TAT and comprises the sequence KKRRQRRR (SEQ ID NO: 105). In another embodiment, the protein transduction domain is Pep-1 and comprises the sequence KETWWETWWTEWSQPKKKRKV (SEQ ID NO: 120).

In addition, or alternatively to the above-mentioned protein transduction domains, the nickase (e.g., Cas9 nickase) recombinant protein or nucleic acid and guide RNA(s) of the present disclosure may be coupled to liposomes to further facilitate their delivery into the cells.

Genetic constructs encoding a deaminase in accordance with the present disclosure can be made using either conventional gene synthesis or modular assembly.

In an aspect, the guide RNAs or pegRNA and recombinant Cas nickase fusion proteins of the present disclosure may be used to decrease toxic amyloidogenic Aβ peptides production. In another aspect, the guide RNAs or pegRNA and deaminase or reverse transcriptase recombinant protein of the present disclosure may be used to decrease APP cleavage at the beta secretase site (e.g., between amino acid 671 and 672 of APP) thereby inhibiting completely or partially the production of amyloidogenic Aβ peptides. As used herein, the expression “decreasing” in “decreasing the expression of amyloidogenic Aβ peptides in a cell” is meant to include circumstances where, in the absence of a guide RNA and of a deaminase protein of the present disclosure, the amyloidogenic Aβ peptides are expressed at certain amount (baseline amount), which is decreased in their presence. It comprises decreasing/reducing/inhibiting the maturation (cleavage) into amyloidogenic Aβ peptides in cells completely or partially. The cell may be a cell expressing a normal level of APP or amyloidogenic Aβ peptides or an abnormal/higher level of amyloidogenic Aβ peptides (as compared to normal conditions).

In an embodiment, the guide RNA or pegRNA and the deaminase or reverse transcriptase of the present disclosure may be used to decrease the production of amyloidogenic Aβ peptides.

In an embodiment, the present disclosure relates to a method of decreasing amyloidogenic Aβ peptide expression, production or accumulation in a subject in need thereof comprising administering to the subject an effective amount of a guide RNA, a recombinant deaminase or reverse transcriptase protein of the present disclosure in order to introduce one or more genetic modifications in a target gene (e.g., APP, PS-1 or PS-2). In an embodiment, the recombinant deaminase or reverse transcriptase protein and guide RNA or pegRNA are specifically formulated for crossing the plasma membrane and reaching the nucleus. In an embodiment, the present disclosure provides a composition comprising a recombinant deaminase or reverse transcriptase protein and a guide RNA or pegRNA of the present disclosure together with a pharmaceutically acceptable carrier. In an embodiment, the method of the present disclosure corrects a mutation present in a target gene, which is associated with increased risk of developing Alzheimer's disease. In an embodiment, the mutation increases the expression or maturation of APP into toxic Aβ peptides. In another embodiment, the method of the present disclosure introduces a modification in a target gene or mRNA, which protects against Alzheimer's disease (i.e., reduces the risk of developing Alzheimer's disease). In an embodiment, the modification decreases the production of amyloidogenic Aβ peptides by modifying one or more of the beta and gamma secretases cleavage sites in the APP protein, thereby reducing amyloidogenic Aβ peptide secretion. In an embodiment the modification is a modification at amino acid 673 or 684 or other amino acids close to that position of the APP protein, which reduces cutting by the beta-secretase. In an embodiment, the modification replaces an alanine at position 673 with a threonine or a valine. In another embodiment, the modification replaces a histidine at position 684 with an arginine.

### Optimization of Codon Degeneracy

Because several site-specific nuclease proteins, such as Cas9, are normally expressed in bacteria, it may be advantageous to modify their nucleic acid sequences for optimal expression in eukaryotic cells (e.g., mammalian cells). This has been done for the embodiment of the Cas9 nuclease and the deaminase proteins of the present disclosure described herein.

Codon degeneracy may also be used to distinguish two nucleic acids encoding for the same protein. For example, the donor/patch nucleic acid sequence of the present disclosure may comprise one or more modifications with respect to the wild type targeted gene sequence, which do not translate into modifications at the amino acid level.

Accordingly, the following codon chart (Table 2) may be used, in a site-directed mutagenic scheme, to produce nucleic acids encoding the same or slightly different amino acid sequences of a given nucleic acid:

TABLE 2
Codons encoding the same amino acid

| Amino Acids | | | | | | | Codons | | | |
|---|---|---|---|---|---|---|---|---|---|---|
| Alanine Cysteine | Ala | Cys | A | C | GCA | GCC | GCG | GCU | | |
| Aspartic acid | Asp | | D | | UGC | UGU | | | | |
| | | | | | GAC | GAU | | | | |
| Glutamic acid | Glu | | E | | GAA | GAG | | | | |
| Phenylalanine | Phe | | F | | UUC | UUU | | | | |
| Glycine | Gly | | G | | GGA | GGC | GGG | GGU | | |
| Histidine | His | | H | | CAC | CAU | | | | |
| Isoleucine | Ile | | I | | AUA | AUC | AUU | | | |
| Lysine | Lys | | K | | AAA | AAG | | | | |
| Leucine | Leu | | L | | UUA | UUG | CUA | CUC | CUG | CUU |
| Methionine | Met | | M | | AUG | | | | | |
| Asparagine | Asn | | N | | AAC | AAU | | | | |
| Proline | Pro | | P | | CCA | CCC | CCG | CCU | | |
| Glutamine | Gln | | Q | | CAA | CAG | | | | |
| Arginine | Arg | | R | | AGA | AGG | CGA | CGC | CGG | CGU |
| Serine | Ser | | S | | AGC | AGU | UCA | UCC | UCG | UCU |
| Threonine | Thr | | T | | ACA | ACC | ACG | ACU | | |
| Valine | Val | | V | | GUA | GUC | GUG | GUU | | |
| Tryptophan | Trp | | W | | UGG | | | | | |
| Tyrosine | Tyr | | Y | | UAC | UAU | | | | |

### Sequence Similarity

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “homologous” to another sequence if the two sequences are substantially identical and the functional activity of the sequences is conserved (as used herein, the term “homologous” does not infer evolutionary relatedness, but rather refers to substantial sequence identity). Two nucleic acid sequences are considered substantially identical if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%. For the sake of brevity, the units (e.g., 66, 67 . . . 81, 82 . . . 91, 92% . . . ) have not systematically been recited but are considered, nevertheless, within the scope of the present disclosure.

Substantially complementary nucleic acids are nucleic acids in which the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, at least 95%, at least 96%, at least 98% or at least 99%. Optimal alignment of sequences for comparisons of identity may be conducted using a variety of algorithms, such as the local homology algorithm of Smith and Waterman³⁸, the homology alignment algorithm of Needleman and Wunsch³⁹, the search for similarity method of Pearson and Lipman⁴⁰, and the computerized implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al.⁴¹ (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighborhood word score threshold. Initial neighborhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridization to filter-bound sequences under moderately stringent conditions may, for example, be performed in 0.5 M NaHPO4, 7% sodium dodecyl sulfate (SDS), 1 mM EDTA at 65° C., and washing in 0.2×SSC/0.1% SDS at 42° C. (64). Alternatively, hybridization to filter-bound sequences under stringent conditions may, for example, be performed in 0.5 M NaHPO4, 7% SDS, 1 mM EDTA at 65° C., and washing in 0.1×SSC/0.1% SDS at 68° C. (64). Hybridization conditions may be modified in accordance with known methods depending on the sequence of interest. Generally, stringent conditions are selected to be about 5° C. lower than the thermal melting point for the specific sequence at a defined ionic strength and pH.

In another aspect, the invention further provides one or more nucleic acids encoding the above-mentioned Cas or deaminase recombinant protein and guide RNA sequences. The invention also provides a vector comprising one or more of the above-mentioned nucleic acids. In an embodiment, the vector further comprises a transcriptional regulatory element operably linked to the above-mentioned nucleic acid. A first nucleic acid sequence is “operably-linked” with a second nucleic acid sequence when the first nucleic acid sequence is placed in a functional relationship with the second nucleic acid sequence. For instance, a promoter is operably linked to a coding sequence if the promoter affects the transcription or expression of the coding sequences. Generally, “operably-linked” DNA sequences are contiguous and, where necessary to join two protein coding regions, in reading frame. However, since, for example, enhancers generally function when separated from the promoters by several kilobases and intronic sequences may be of variable lengths, some polynucleotide elements may be operably-linked but not contiguous. “Transcriptional regulatory element” is a generic term that refers to DNA sequences, such as initiation and termination signals, enhancers, and promoters, splicing signals, polyadenylation signals, which induce or control transcription of protein coding sequences with which they are operably-linked.

As indicated above, guide RNAs or pegRNA and Cas or deaminase or reverse transcriptase recombinant nucleic acids of the present disclosure may be delivered into cells using various viral vectors. Accordingly, preferably, the above-mentioned vector is a viral vector for introducing the guide RNA or pegRNA and/or Cas and/or deaminase or reverse transcriptase nucleic acid encoding the Cas9, the deaminase or reverse transcriptase sequence of the present disclosure in a target cell (preferably a neuron). Non-limiting examples of viral vectors include retrovirus, lentivirus, Herpes virus, adenovirus or adeno Associated Virus, as well known in the art. Herpes virus, adenovirus, Adeno-Associated virus and lentivirus derived viral vectors have been shown to efficiently infect neuronal cells. Preferably, the viral vector is episcopal and not cytotoxic to cells. In an embodiment, the viral vector is an AAV or a Herpes virus. The Cas9 nickase, the deaminase or reverse transcriptase genes or the Cas9 nickase and/or the deaminase or reverse transcriptase protein may also be delivered to the cells by virus like particles (VLP), exosomes or other extra-cellular vesicles such as exosomes.

In yet another aspect, the present disclosure provides a cell (e.g., a host cell, a recombinant cell) comprising the modified targeted gene or mRNA. The invention further provides a recombinant expression system, vectors and host cells, such as those described above, for the expression/production of a recombinant protein, using for example culture media, production, isolation and purification methods well known in the art.

In another aspect, the present disclosure provides a composition (e.g., a pharmaceutical composition) comprising the above-mentioned guide RNA or pegRNA and a recombinant nickase (e.g., Cas9) fused with a deaminase or reverse transcriptase nucleic acid or protein. In an embodiment, the composition comprises the above-mentioned viral vectors, virus like particles (VLP), exosomes or other extra-cellular vesicles for targeting the guide RNA, nickase (e.g., Cas9n), the REPAIRv2 deaminase or the ADAR2 deaminase into a cell. In an embodiment, the cell is a neuronal cell. In an embodiment, the composition further comprises one or more pharmaceutically acceptable carriers, excipients, and/or diluents.

As used herein, “pharmaceutically acceptable” (or “biologically acceptable”) refers to materials characterized by the absence of (or limited) toxic or adverse biological effects in vivo. It refers to those compounds, compositions, and/or dosage forms, which are, within the scope of sound medical judgment, suitable for use in contact with the biological fluids and/or tissues and/or organs of a subject (e.g., human, animal) without excessive toxicity, irritation, allergic response, or other problem or complication, commensurate with a reasonable benefit/risk ratio.

The present disclosure further provides a kit or package comprising the above-mentioned guide RNA or pegRNA and recombinant nickase and/or deaminase or reverse transcriptase protein or composition, together with instructions for reducing amyloidogenic Aβ peptide production levels (expression or maturation) in a cell or for the treatment of Alzheimer's disease or age-related cognitive decline.

The present disclosure is illustrated in further details by the following non-limiting examples.

### EXAMPLES

### Example 1: Effect of A673 Mutation on Aβ Peptide Formation in APP Containing FAD Mutation(s)

Our first experiment aimed to verify whether the Icelandic mutation (A673T) reduced the formation of Aβ40 and Aβ42 peptides when a FAD mutation was also present in the APP gene. This was tested by constructing various plasmids containing the APP gene with one to three FAD mutations with or without an additional Icelandic mutation. These plasmids were then transfected in 293T cells or in SH-SY5Y cells and the concentration of Aβ40 and Aβ42 peptides was measured in the culture medium 3 days later.

The plasmid used as a backbone was pcDNA6N5-His from Invitrogen Inc. A cDNA APP695 was inserted by ligation between the Kpn1 and the Xba1 cutting sites. This plasmid was then mutated with New England Biology Inc. (NEB) mutagenesis Q5 kit in order to create a single point mutation responsible of a Familial Form of Alzheimer disease (FAD). Several mutations in APP exons 16 and 17 known to induce a FAD by increasing the Alzheimer pathogenicity were created: KM670/671NL⁴², A673V⁴³, H677R⁴⁴, D678H (Chen et al., 2012), D678N⁴⁵, E682K⁴⁶, A692G⁴⁷, E693del⁴⁸, E693G⁴⁹, D694N⁵⁰, A713T⁵¹, T714A⁵², T714I⁵³, V715A⁵⁴, V715M⁵⁵, I716F⁵⁶, I716M⁵⁷, I716T⁵⁸, I716V⁵⁹, V717F⁶⁰, V717G⁶¹, V717I⁶², V717L⁶³, T719P⁶⁴, M722K⁶⁵, L723P⁶⁶, K724N⁶⁷.

Plasmids containing 2 or 3 FAD mutations were also created because several mouse models of AD contained these combinations of mutations.

In order to determine the protective effect of the Icelandic mutation A673T, another set of APP cDNA plasmids was also made containing not only one or several FAD mutations but also an in additional Icelandic mutation (A673T).

Transfection in SH-SY5Y or in 293T Cells of Plasmids Coding for the APP695 cDNA Containing One or Several FAD Mutations and Containing or not the Icelandic Mutation.

The transfection reagent (Lipofectamine2000™) and Opti-MEM-1™ culture media) were purchased from Life Technologies Inc.

The day before the transfection, 100,000 293T or SH-SY5Y cells were seeded per well in a 24 well plates in DMEM (DMEM F12 for SH-SY5Y cells) medium supplemented with 10% FBS and antibiotics (penicillin/streptomycin 1×). The following morning, the culture medium was changed for 500 μl of DMEM medium (DMEM F12 for SH-SY5Y cells) supplemented with 10% FBS without antibiotics. The plate was incubated at 37° C. in the incubator for the time required to prepare the transfection solution. For the transfection, solutions A and B were first prepared. Solution A contained 48 μl of Opti-MEM™ and 2 μl of Lipofectamine™ 2000 for a final volume of 50 μl. Solution B was prepared as follows: a volume of DNA solution containing 150 ng to 800 ng of DNA was mixed with a volume of Opti-MEM™ to obtain a final volume of 50 μl. For each co-transfection, various amounts of the plasmids were used. Solutions A and B were then mixed by up and down movement and incubated at room temperature for 20 minutes. 100 μl of the mixed solution were then added to each well. The plate was let in the CO₂ incubator for a period of 4 to 6 hours. The medium was changed by 1 ml of DMEM (DMEM F12 for SH-SY5Y cells) supplemented with 10% FBS and antibiotics. The plate was incubated for 72 hours in the CO₂ incubator before extraction of genomic DNA. The supernatants were harvested, a protease inhibitor (1 mM PMSF, phenylmethane sulfonyl fluoride or phenylmethylsulfonyl fluoride) was added and this was stored at −80° C.

The concentrations of Aβ40 and Aβ42 peptides were measured with Meso Scale Discovery Inc. (MSD) Neurodegenerative Disease Assay kit (using the 6E10 antibody for preliminary immuno-precipitation). The experiment results (Table 3) showed that the Aβ40 and Aβ42 peptide concentrations in the supernatants were decreased for a majority of the cells transfected with a plasmid coding for the APP cDNA containing not only one or several FAD mutations but also the Icelandic mutation (A673T), compared to cells transfected either with the wild type APP cDNA or a APP cDNA containing only one or several FAD mutations. Thus, the A673T mutation reduces the formation of Aβ peptides even in the presence of a FAD mutation and thus should prevent or slow-down the progression of Alzheimer disease in patients with a FAD mutation. The significance was determined by a Sidak's ANOVA test. P value style *p<0.0332, **p<0.0021, ***p<0.0002, ****p<0.0001.

TABLE 3

| | Aβ42% | | | Aβ40% | | |
|---|---|---|---|---|---|---|
| EOAD mutation | Decrease | S.D. | Sign. | Decrease | S.D. | Sign. |
| V717I (London) | −63 | 1.8 | **** | −80 | 11.8 | **** |
| V717G | −55 | 0.6 | **** | −24 | 1.8 | ns |
| D694N (Iowa) | −47 | 5.3 | ns | −56 | 5.7 | ns |
| Wild-type | −45 | 6.5 | * | −65 | 13.7 | * |
| T714I (Austrian) | −42 | 1.7 | **** | −33 | 0.2 | ns |
| A692G (Flemish) | −41 | 7.8 | ns | −40 | 7.0 | **** |
| V715A (German) | −40 | 2.5 | **** | −36 | 3.4 | * |
| M722K | −38 | 4.2 | *** | −31 | 0.2 | ns |
| V715M (French) | −36 | 3.2 | **** | −26 | 6.2 | ns |
| V717L | −36 | 11.4 | ns | −40 | 9.1 | ns |
| I716F (Iberian) | −35 | 15.3 | ns | −51 | 27.2 | ns |
| K687N | −32 | 3.8 | ns | −28 | 7.7 | ns |
| T719P | −30 | 1.5 | *** | −9 | 11.1 | ns |
| E682K (Leuven) | −30 | 6.3 | **** | −31 | 2.4 | **** |
| E693del (Osaka) | −28 | 3.7 | ns | −44 | 6.7 | ns |
| V717F | −28 | 1.3 | * | −16 | 0.5 | ns |
| I716M | −23 | 2.7 | ** | −29 | 3.9 | ** |
| NL/F/G | −20 | 12.9 | ns | −64 | 33.1 | ns |
| T714A (Iranian) | −19 | 4.8 | ns | −10 | 4.8 | ns |
| A713T | −18 | 0.5 | ns | −26 | 0.6 | ns |
| E693K (Italian) | −18 | 0.2 | ns | −32 | 6.2 | ns |
| I716V (Florida) | −17 | 4.9 | ns | −19 | 1.9 | ns |
| KM670/671NL | −10 | 7.9 | ns | −1 | 10.4 | ns |
| (Swedish) | | | | | | |
| K724N (Belgian) | −5 | 0.2 | ns | 3 | 0.7 | ns |
| D678N (Tottori) | −3 | 4.0 | ns | −13 | 11.7 | −13 |
| E693Q (Dutch) | −2 | 4.4 | ns | −4 | 1.0 | ns |
| E693G (Arctic) | 10 | 46.8 | ns | 20 | 41.8 | ns |
| L723P (Australian) | 15 | 5.8 | ns | 6 | 1.8 | ns |
| D678H (Taiwanese) | 21 | 20.3 | ns | 22 | 38.8 | ns |
| I716T | 44 | 0.3 | ** | 116 | 0.2 | ns |
| H677R (English) | 102 | 5.0 | ** | 110 | 21.5 | * |
| NL/F | 139 | 14.3 | ns | 64 | 14.8 | * |

S.D.: Standard deviation
ns: no signal

### Example 2: Introduction of A673T Mutation Via Base Editing Using the CRISPRICas9 System

The second experiment aimed to introduce the Icelandic mutation (A673T) in the APP gene by using a site-specific cytidine deaminase fused with a Cas nickase (Cas9n). The alanine codon in position 673 is 5′GCA3′, the antisense codon is thus 5′TGC3′. We aimed to modify the cytidine in the antisense codon into a thymidine thus producing the antisense codon 5′TGT3′. The sense codon will thus be 5′ACA3′ which codes for threonine. For this experiment, 293T or SH-SY5Y cells were co-transfected with a plasmid coding for a Cas9n/Cytidine deaminase and the pBSU6 plasmid coding for a guide RNA targeting a specific sequence of the APP gene.

The site-specific cytidine deaminases are fusion enzymes, which include a mutated Cas9 nuclease (D10A) to generate a Cas9 nickase (Cas9n). This Cas9n may be fused with various deaminases (either the APOBEC1 or the Activation-induced cytidine deaminase (Target-AID or CDA1) and various spacer lengths (FIG. 4). The resulting hybrid enzymes designated as BE3, BE4 and Target-AID. For our experiments, we have also generated genes coding for several new hybrid Cas9 deaminases including not only the Cas9 nickase from Streptococcus pyogenes, but also the Cas9 nickase from Staphylococcus aureus and a variant of the SaCas9 nickase named KKH SaCas9, which reacts with NNNRRT PAMs⁶⁸.

Modifications of the SpCas9 nuclease (designated under the names VQR and EQR) have previously been produced to detect various PAMs⁶⁹. The VQR variant robustly reacting with NGAN PAMs and the EQR variant being more specific for an NGAG PAM. Various Cas9 cytidine deaminases have also been previously described^{31,70}. A variant of SaCas9 called SaCas9-KKH has also been produced^{68, 71}. We have thus also produced additional hybrid enzymes including these SpCas9 and SaCas9 variants. The list of the deaminases that we have produced and tested is the following: BE3_SpCas9n_{VQR}, BE3_SpCas9n_{EQR}, BE3_SaCas9n_{KKH}, YE1-BE3, YE1-BE3_SpCas9n_{VQR}, YE1-BE3_SpCas9n_{EQR}, YE1-BE3_SaCas9n_{KKH}, BE4-Gam_SpCas9n_{VQR}, BE4-Gam_SpCas9n_{EQR}, Target-AID SpCas9n_{VQR}, Target-AID SpCas9n_{EQR}, Target-AID SaCas9n_{KKH}, hADAR2, REPAIRv2, ABE6.3, ABE7.8, ABE7.9, ABE7.10. Two spacer lengths, either 17 bp to 22 bp, were tested in order to influence the conversion window. One or two guide RNAs were inserted in a modified pBSU6 plasmid.

1) BE3_SpCas9n_{VQR} (SEQ ID NOs: 3 and 4) is a variant of BE3, which contains a SpCas9n protein with 3 mutated amino acids D1135V/R1335Q/T1337R. This SpCas9n_{VQR} enzyme was produced by Kleinstiver et al.⁶⁹. The gene for this enzyme was available at Addgene Inc. as pBK-VQR-BE3 (#85171).

2) BE3_SpCas9n_{EQR} (SEQ ID NOs: 5 and 6) is a variant of BE3, which contains a SpCas9n protein with 3 mutated amino acids D1135E/R1335Q/T1337R. This SpCas9n_{EQR} enzyme was done by Kleinstiver et al⁶⁹. The gene for this enzyme was available at Addgene Inc. as pBK-EQR-BE3 (#85172).

3) BE3_SaCas9n_{KKH} (SEQ ID NOs: 7 and 8) is a variant of BE3, which contains a SaCas9n protein (from Staphylococcus aureus) with 3 mutated amino acids E782K/N968K/R1015H. This mutated SaCas9n_{KKH} enzyme was done by Kleinstiver et al⁶⁸. The gene for this enzyme was available at Addgene Inc as pJL-SaKKH-BE3 (#85170).

4) YE1-BE3 (SEQ ID NOs: 9 and 10) is a construct variant of BE3, which contain 2 mutated amino acid (W90Y/R126E) in the rAPOBEC1 sequences. The gene for this enzyme was available at Addgene Inc. as pBK-YE1-BE3 (#85174). We used this plasmid to construct all other variants containing the YE1 rAPOBEC1 deaminase.

5) YE1-BE3_SpCas9n_{VQR} (SEQ ID NOs: 11 and 12) is a variant of the YE1-BE3 that was made in our laboratory. rAPOBEC1 version in plasmid pBK-VQR-BE3 was replaced by the YE1 version from plasmid YE1-BE3.

6) YE1-BE3_SpCas9n_{EQR} (SEQ ID NOs: 13 and 14) is a variant of the YE1-BE3 that was made in our laboratory. rAPOBEC1 version in plasmid pBK-EQR-BE3 was replaced by the YE1 version from plasmid YE1-BE3.

7) YE1-BE3_SaCas9n_{KKH} (SEQ ID NOs: 15 and 16) is a variant of the BE3_SaCas9n_{KKH} that was made in our laboratory. rAPOBEC1 version in plasmid BE3_SaCas9n_{KKH} was replaced by the YE1 version from plasmid YE1-BE3.

8) BE4-Gam_SpCas9n_{VQR} (SEQ ID NOs: 17 and 18) is a variant of the BE4-Gam available at Addgene Inc. #100806 that was made in our laboratory by PCR mutagenesis to create VQR mutation in the SpCas9n gene.

9) BE4-Gam_SpCas9n_{EQR} (SEQ ID NOs: 19 and 20) is a variant of the BE4-Gam available at Addgene Inc #100806. This variant was made in our laboratory by PCR mutagenesis to create EQR mutation in the SpCas9n gene.

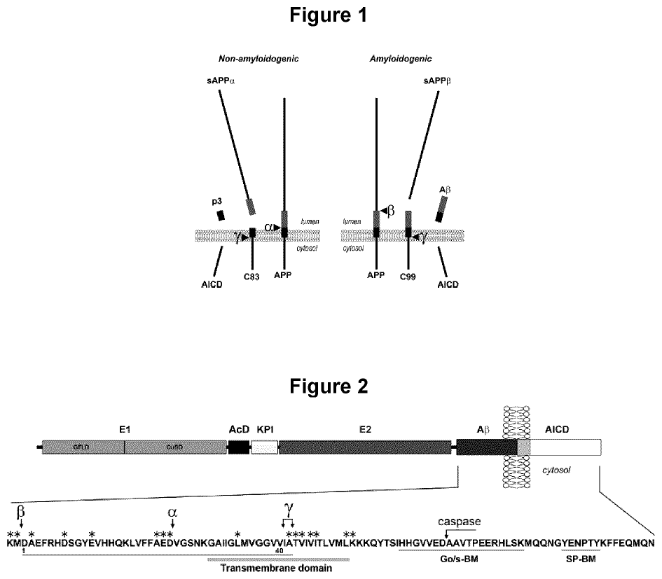

10) Target-AID SpCas9n_{VQR} (SEQ ID NOs: 21 and 22) is a variant of the Target-AID enzyme described by Komor et al.¹⁰. As shown in FIG. 1, the original Target-AID enzyme contains the SpCas9n(D10A), a 105 amino acids linker, CDA1 (i.e., the activation induced cytidine deaminase (AID) that was modified by Nishida et al.⁷⁰), a 11 amino acids linker and the UGI. The original plasmid, named nCas9-PmCDA1-UGI available at Addgene Inc. #76620 was modified by PCR mutagenesis to create the VQR version of the SpCas9n.

11) Target-AID SpCas9n_{EQR}(SEQ ID NOs: 23 and 24) is a variant of the Target-AID enzyme described by Komor et al.¹⁰. The original Target-AID enzyme contains the SpCas9n(D10A), a 105 amino acids linker, CDA1 (i.e., the activation induced cytidine deaminase (AID) that was modified by Nishida et al.⁷⁰), a 11 amino acids linker and the UGI. The original plasmid, named nCas9-PmCDA1-UGI and guide RNA(HPRT)(Target-AID) available at Addgene Inc. #76620 was modified by PCR mutagenesis to create the EQR version of the SpCas9n.

12) Target-AID SaCas9n_{KKH} (SEQ ID NOs: 25 and 26) is a variant of the original plasmid called Target-AID available at Addgene Inc. #76620. The SpCas9n was replaced by SaCas9n_{KKH}. Briefly, the SpCas9n was removed by cutting the plasmid with two restriction enzymes. The SpCas9n of the #76620 plasmid was replaced by the SaCas9n_{KKH}.

13) hADAR2 (SEQ ID NOs: 27 and 28) is a member of the adenosine deaminase acting on RNA (ADAR) family of enzymes, which mediates endogenous editing of transcripts via hydrolytic deamination of adenosine to inosine, a nucleobase that is functionally equivalent to guanosine in translation and splicing. There are two functional human ADAR orthologs, ADAR1 and ADAR2, which consist of N-terminal double stranded RNA-binding domains and a C-terminal catalytic deamination domain.

14) REPAIRv2 (SEQ ID NOs: 29 and 30) is was produced by Cox et al.¹⁶ using rational mutagenesis scheme to improve the specificity of dCas13b-ADAR2DD fusions to generate an enzyme with more than 919-fold higher specificity. Addgene plasmid #pC0055).

15) ABE6.3 (SEQ ID NOs: 31 and 32), ABE7.8 (SEQ ID NOs: 33 and 34), ABE7.9 (SEQ ID NOs: 35 and 36), ABE7.10 (SEQ ID NOs: 37 and 38) are various DNA adenosine deaminase enzymes that were produced by Gaudelli et al.²³ by mutagenesis of a transfer RNA adenosine deaminase. The coding plasmids were obtained from AddGene inc. (respectively #102916, #102917, #102918 and #102919).

Various SpCas9 VQR/EQR guide RNAs targeting
sequences of 17 to 22 nucleotides were tested:
(SEQ ID NO: 53)
ATTCTGCATCCATCTTCACTTCAGAG
(SEQ ID NO: 54)
TTCTGCATCCATCTTCACTTCAGAG
(SEQ ID NO: 55)
TCTGCATCCATCTTCACTTCAGAG
(SEQ ID NO: 56)
CTGCATCCATCTTCACTTCAGAG
(SEQ ID NO: 57)
TGCATCCATCTTCACTTCAGAG
(SEQ ID NO: 58)
GCATCCATCTTCACTTCAGAG
Various SaCas9 guide RNAs were tested:
(SEQ ID NO: 59)
ATTCTGCATCCATCTTCACTTCAGAGAT
(SEQ ID NO: 60)
TTCTGCATCCATCTTCACTTCAGAGAT
(SEQ ID NO: 61)
TCTGCATCCATCTTCACTTCAGAGAT
(SEQ ID NO: 62)
CTGCATCCATCTTCACTTCAGAGAT
(SEQ ID NO: 63)
TGCATCCATCTTCACTTCAGAGAT
(SEQ ID NO: 64)
GCATCCATCTTCACTTCAGAGAT

### Deamination Results

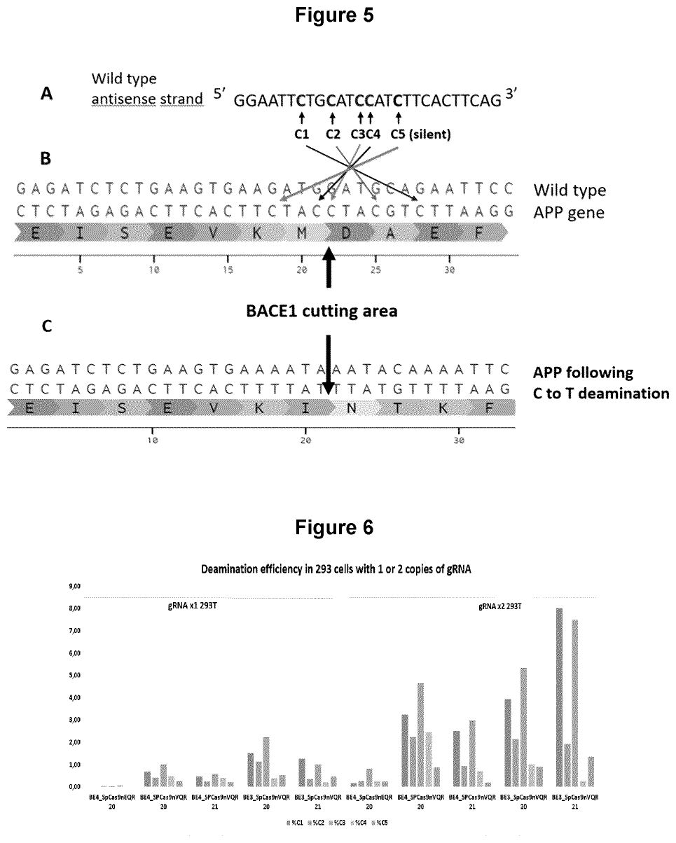

FIG. 5 upper section illustrates DNA sequence of a wild-type APP exon 16 sense strand and antisense strand APP. The lower section of the figure illustrates the fully deaminated (C to T conversion) of the 5 cytidines (C1 to C5) located within the conversion window of the cytidine deaminase APOBEC1 and AID. The deamination of the cytidine C1 in the antisense strand modifies the glutamic acid codon (GAA) in position 674 into a lysine codon (MA). The deamination of the cytidine C2 in the antisense strand changes the alanine codon (GCA) in position 673 into a threonine codon (ACA), thus inducing the Icelandic A673T mutation. The deamination of C3 in the antisense strand changes the aspartic acid codon (GAT) in position 672 into an asparagine codon (AAT). The deamination of the C4 nucleotide changes the methionine codon (ATG) in position 671 into an isoleucine codon (ATA). The deamination of the C5 nucleotide is a silent mutation that does not modify the resulting amino acid (lysine) in position 670.

The objective of the proposed Alzheimer treatment is to deaminate the highest percentage of C2 and the lowest percentages of the other four C to generate the A673 Icelandic mutation.

### Experiment Description

100,000 293T cells or SH-SY5Y cells were deposited in each well of a 24 wells plate. These cells were transfected with a plasmid coding for one Cas9-deaminase and one guide RNA. The cells were incubated at 37° C. Cells were detached 72 hours later by performing up and down movements in 1 ml culture medium with a pipette. These cells were transferred in an Eppendorf tube and centrifuge at 8000 RPM for 10 minutes. The supernatant medium was carefully removed without disturbing the cell pellets. These cell pellets were washed once with 1 ml of HBSS solution and centrifuged at 8000 RPM for 10 minutes. The HBSS was then carefully removed without disturbing the cell pellets. The cells were lysed with 100 μl of lysis buffer containing 1% Sarkosyl and 0.5M EDTA pH8 supplement with 10 μl of proteinase K solution (20 mg/ml). These tubes were incubated at 56° C. for 15 minutes. 400 μl of 50 mM Tris pH8 were then added to each tube. Next, 500 μl of a mixture of phenol:chloroform:isoamyl alcohol (respectively 25:24:1) was added. The tubes were centrifuged at 16 000 RPM for 2 minutes. The aqueous upper phase was transferred to a new tube. 50 μl of 5 M NaCl was added to each tube and mixed thoroughly. One (1) ml of ice-cold ethanol 100% was added to each tube and mixed for genomic DNA precipitation. The tubes were centrifuged at 16000 RPM for 7 minutes and ethanol was carefully removed to avoid disturbing the DNA pellets. These DNA pellets were washed once with 400 μl ethanol 70%. The tubes were centrifuged at 16000 RPM for 5 minutes and ethanol was removed to permit to dry the DNA pellet rapidly by lyophilization. The DNA was solubilized in 50-100 μl of sterile water and stored at −20° C. until quantification was performed. The DNA solutions were dosed at 260 nm with a spectrophotometer.

### PCR Amplification

For PCR amplification the following mix was used: genomic DNA (25 ng/μl, 2 μl), 5×HF Phusion™ buffer (10 μl), dNTP 10 mM (1 μl), forward primer (100 ng/μl, 1 μl), reverse primer (100 ng/μl, 1 μl), water (34.75 μl) and Phusion™ enzyme (2U/μl, 0.25 μl). PCR amplification was as follows: denaturation at 98° C. for 30 sec. followed by (98° C.-10 sec, 60° C.-20 sec. and 72° C. 45 sec. to 1 min) for 35 cycles; and 72° C. for 5 minutes as a final elongation cycle.

### Surveyor Nuclease Assay for Confirmation of Genome Modification

After transfection, SH-SY5Y or 293T cells were incubated at 37° C. for 72 hrs and genomic DNA was extracted as described above. The genomic region of exon 16 of the APP gene was PCR amplified with different forward primers Fb, Fc and different reverse primers Rb, Rc using Phusion™ DNA Polymerase (New England Biolabs Inc.) as described in genomic PCR amplification section. After the PCR amplification, 20 μl of unpurified PCR products were heated at 95° C. for 5 minutes and slowly cooled down (5° C. per 30 seconds) to 25° C. using a thermocycler. After the formation of heteroduplexes, 1 μL of surveyor enzyme (Transgenomics Inc. Omaha, Nebr., USA) and 1 μL of enhancer in Phusion™ HF Buffer (NEB Inc.) were added with to each tube to obtain a 1× final concentration. The mixes were incubated at 42° C. for 25 to 60 minutes. 5 μl of loading buffer were added to each tube. All Surveyor analyses were done on 2% agarose gels containing RedSafe™ Nucleic Acid staining solution (Froggabio Inc., Toronto, ON, Canada) for UV visualization.

### Deep Sequencing Analysis

Deep sequencing samples were prepared by a PCR reaction with special primers containing a bar code sequence (BCS) sequences to permit the subsequent sequencing.

BCS1:
(SEQ ID NO: 121)
ACACTGACGACATGGTTCTACA
BCS2:
(SEQ ID NO: 122)
TACGGTAGCAGAGACTTGGTCT

DNA sequences were analyzed with the Illumina sequencer. Roughly 10000 reads were obtained per sample.

### Results

The deep sequencing results permitted to determine the percentage of deamination (i.e., a cytidine changed into a thymidine) obtained for each cytidine (C1 to C5) present in the target window. As indicated in FIG. 6, the best deamination percentages were obtained with the Cas9-VQR-BE3 enzyme with a guide RNA targeting 20 nucleotides. Higher percentages of deamination were obtained when 2 sequences coding for the guide RNA were present in the plasmid. However, with this enzyme, the C1 nucleotide was deaminated more frequently than the C2 nucleotide.

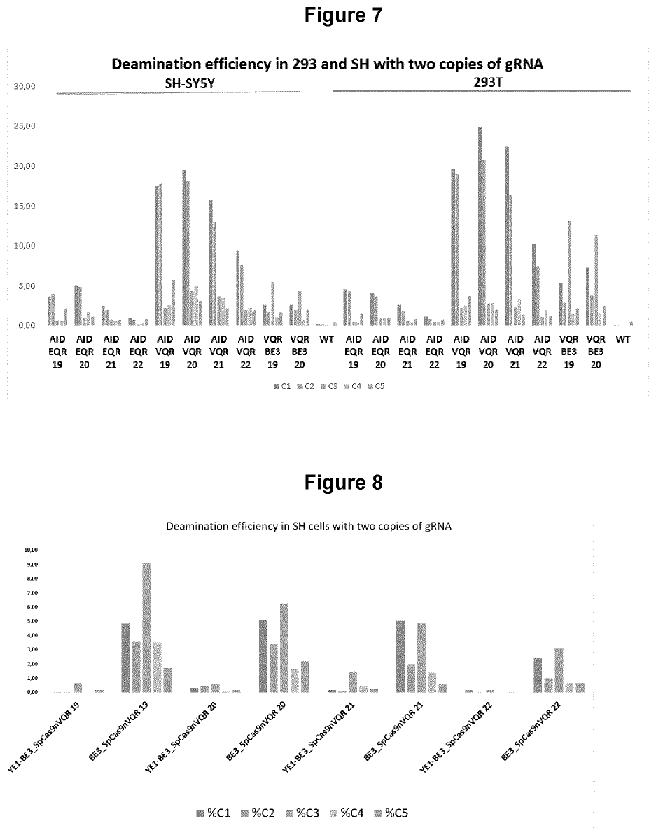

The experiment was thus repeated with various Cas9-deaminases. The Target-AID-Cas9-VQR permitted to deaminate a higher percentage of cytidines (FIG. 7). With this enzyme, the same deamination profile was observed in the 293T cells and in the SH-SY5Y cells. However, a higher deamination percentage of deamination was obtained in the 293T cells, because these cells were transfected more efficiently. The percentage of deamination of C1 and C2 were higher in the SH-SY5Y and the 293T cells with Target-AID-Cas9VQR deaminase than with the other variants (FIG. 7).

The YE1 variant of APOBEC1 contains two mutations W90Y and R126E. We also tested this YE1 variant fused with various SpCas9n. The YE1 variant fused with the SpCas9n variant was reported to create a narrower conversion window⁷². However, this cytidine deaminase variant produced a very low percentage of conversion of cytidine into thymidine in exon 16 of the APP gene (FIG. 8).

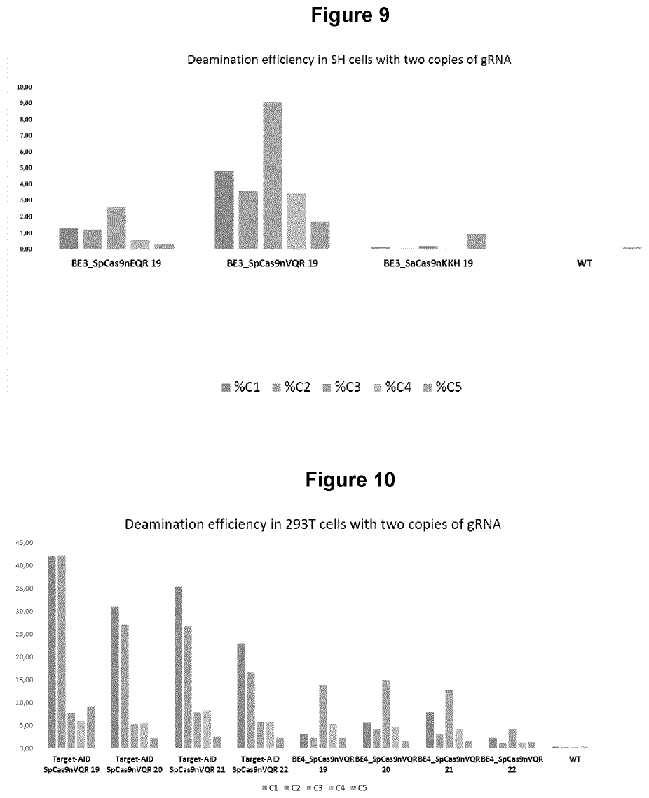

Overall, the EQR variant of SpCas9 and SaCas9-deaminase produced lower deamination rates than the VQR variant of SpCas9 VQR (FIG. 9).

The guide length (i.e., the number of nucleotides to which the guide RNA is binding) also plays an important role in the deamination efficacy and the conversion range. We thus tested guide RNAs binding to 19, 20, 21 or 22 nucleotides. The best deamination of the C2 nucleotide was obtained with the Target-AID Cas9-VQR with a guide RNA binding with 19 nucleotides (FIG. 10).

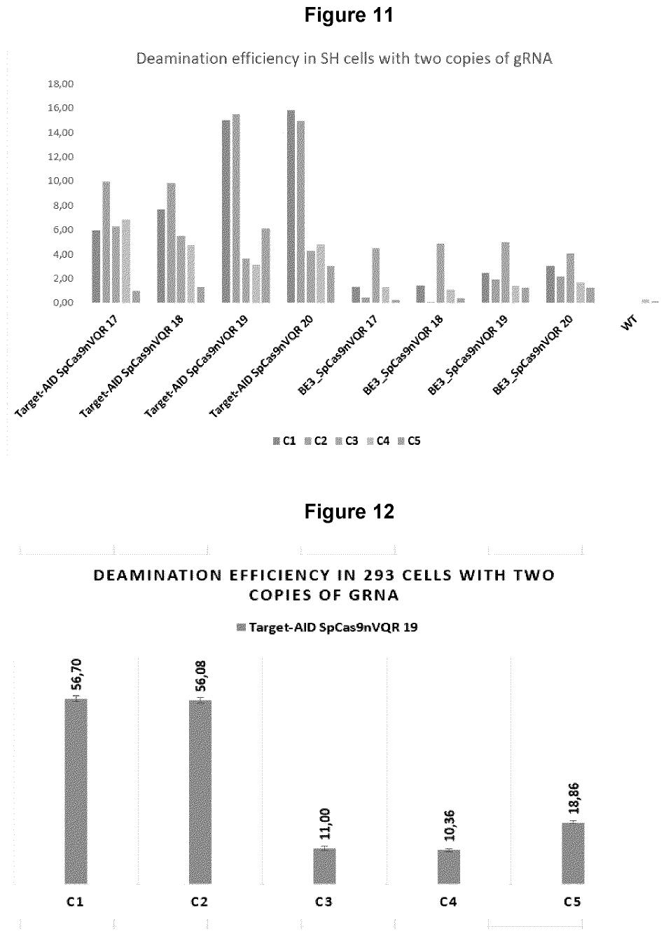

The experiment was repeated with Target-AID-Cas9_{VQR}, BE3-Cas9_{VQR} combined with guide RNA targeting 17, 18, 19 or 20 nucleotides. The highest percentage of deamination of the nucleotides C1 and C2 were obtained with the Target-AID-Cas9_{VQR} used with guide RNAs targeting 19 or 20 nucleotides (FIG. 11). BE3-Cas9VQR (FIG. 11) and BE4-Cas9VQR (FIG. 10) variants showed a different profile than Target-AID variants. Thus BE3-Cas9_{VQR} and BE4-Cas9_{VQR} promote C1 and C3 deamination whereas Target-AID promotes C1 and C2 deamination.

The best results were obtained with the Target-AID-SpCas9n_{VQR} nickase in combination with guide RNA reacting with 19 nucleotides (FIG. 12). Indeed, more than 50% of C1 and C2 were modified into thymines.

### Example 3: Deamination of APP Containing a FAD Mutation

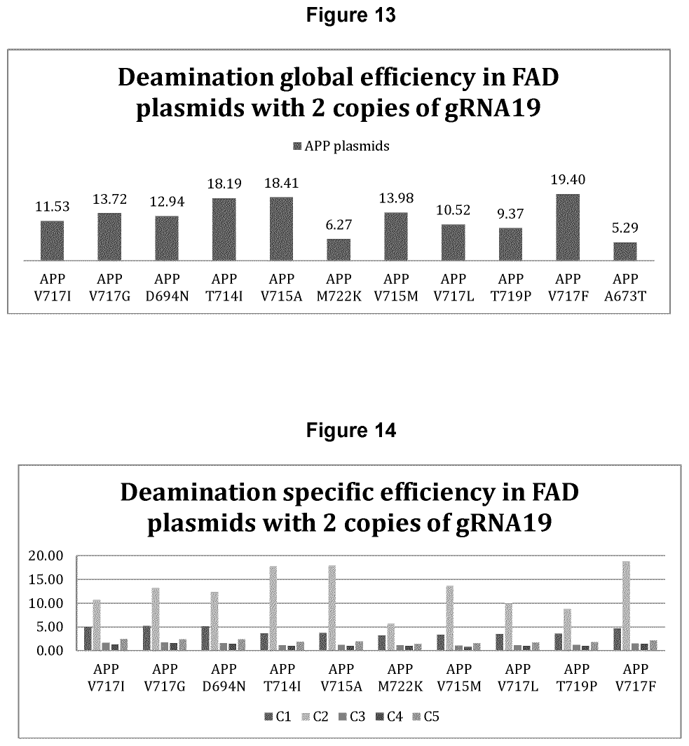

The next experiment was to directly deaminate plasmids coding for APP containing one mutation responsible for a Familial Alzheimer disease (FAD). We selected the 10 FAD mutations for which the presence of the additional A673T mutation led in preliminary experiments to a strong reduction of the formation of Aß42 peptides. SH-SY5Y cells were transfected with one of these APP plasmids containing a FAD mutation, with the Target-AID-SpCas9n_{VQR} plasmid containing two copies of the guide RNA targeting 19 nucleotides. The cell culture supernatant was harvested to analyse with the Meso Scale Discovery (MSD) the concentration of Aß40 and of Aß42 peptides. The genomic DNA and plasmid DNA were also extracted for deep sequencing. The deep sequencing results showed a strong global deamination activity (FIG. 13).

Interestingly, as observed in FIG. 14, we found different deamination profiles with APP plasmids containing different FAD mutations. Indeed, C1 is not as frequently deaminated as C2, which corresponds to our aim of producing the A673T mutation. The ten best FAD mutations showed between 5.3-19.4% deamination of C2, despite the fact that the transfected cells were not selected.

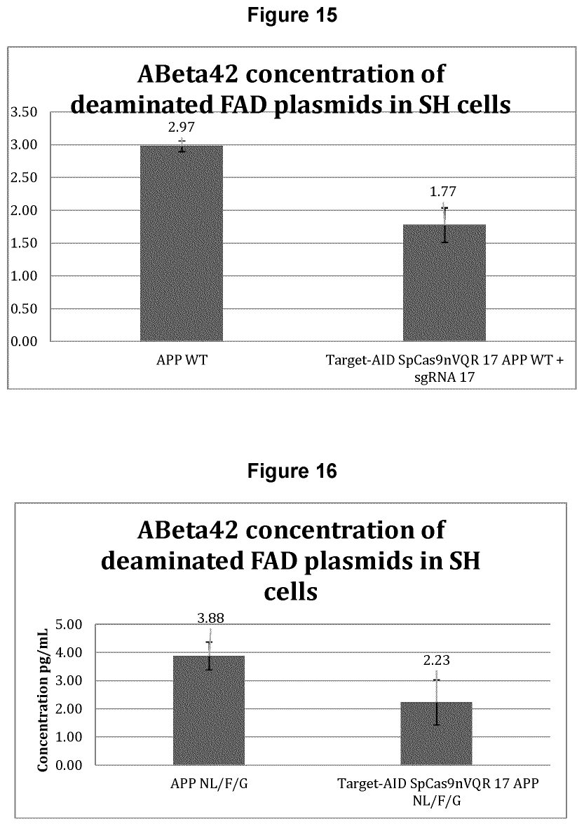

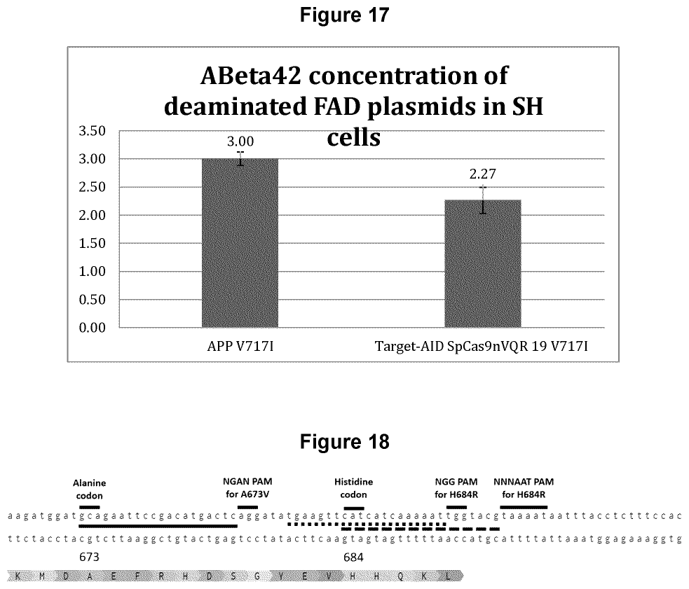

We produced with lentivirus infection different SH-SY5Y cell lines that constitutively express Target-AID-SpCas9n_{VQR} with a guide RNA targeting either 17 (see FIG. 8) or 19 nucleotides. These cells were subsequently transfected with APP plasmids coding for one of the 10 FAD mutations or for the three mutations (the Swedish mutation KM670/671NL, the Arctic mutation E693G and the Beyreuther/Iberian mutation 1716F) present in the NL/G//F Alzheimer mouse model developed by Saito et al.⁷³. This experiment produced very interesting results (FIGS. 15, 16 and 17).

We observed decreases of the Aβ 42 peptide in the culture medium: 40% with plasmids coding for the wild type APP, 43% with an APP plasmid coding for the NL/G/F mutations present in the Alzheimer mouse model and 24% with APP plasmid containing the London mutation (V717I), another Alzheimer mouse model for which we have recently obtained cells from the Remynd company in Belgium.

### Example 4: Inducing the APP A673V Mutation

The alanine codon in position 673 is GCA, it was thus possible to deaminate the cytidine into a thymidine to produce the GTA codon for valine. We have tested different Cas9n-cytidine deaminase enzymes: BE3_SpCas9n, BE4-Gam_SpCas9n and Target AID_SpCas9n to target a NGG PAM in the sense strand. We also tested the BE3_SpCas9n-VQR, BE4-Gam_SpCas9n-VQR, Target-AID_SpCas9n-VQR variants targeting a NGAN PAM in the sense strand.

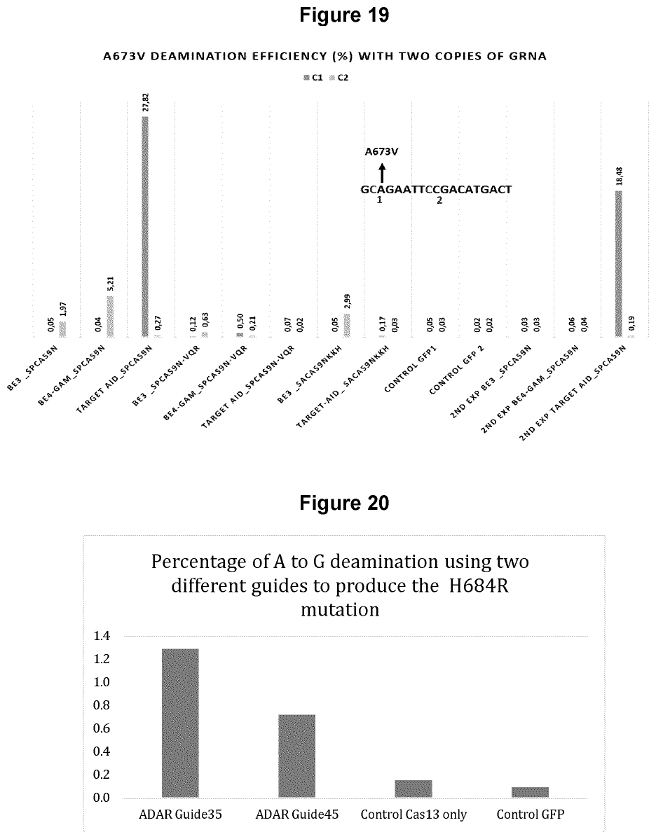

We have thus transfected the human neuroblastoma SH-SY5Y cells with a plasmid coding for each of the deaminases listed above and for a guide RNA targeting the 20 nucleotides 5′ of their respective PAM. The sequences targeted by the guide RNAs are 5′GCAGAATTCCGACATGACTC 3′ (SEQ ID NO: 123) for the NGG PAM (this PAM is indicated in FIG. 18), 5′ATGGATGCAGAATTCCGACA3′ (SEQ ID NO: 124) for the NGAN PAM and 5′ TGCAGAATTCCGACATGACT3′ (SEQ ID NO: 125) for the NNNRRT PAM (see Table 4). The DNA was extracted 3 days later and exon 16 of the APP gene was PCR amplified. Among all the variants, only BE4-Gam_SpCas9n and Target AID_SpCas9n gave positive Surveyor enzyme results. The amplicons were sent for deep sequencing to obtain 10,000 sequences to evaluate the percentage of intended mutation. Table 5 gives the results for Target AID_SpCas9n, which was the most efficient deaminase to modify the first C in the targeted sequence. As indicated in that table 27.82% of the first cytidine located in position 2 of the protospacer was deaminated to a thymidine. The other cytidines in the protospacer sequence were not deaminated compared with the untreated negative control. The results are illustrated in FIG. 19. Thus the deamination of the first C in the protospacer sequence of Target-AID_SpCas9n permits to change the alanine codon (GCA) in position 673 into a GTA codon for valine. This A673V mutation is protective for Alzheimer when it is heterozygote.

TABLE 4
PAM and Protospacer sequences
for different Cas9-deaminases

| | | | SEQ |
|---|---|---|---|
| Deaminase | | Protospacer | ID |
| name | PAM | sequence | NO: |
| BE3_SpCas9n | 5′AGG3′ | 5′ GCAGAATTCC | 126 |
| | | GACATGACTC 3′ | |
| BE4-Gam_ | 5′AGG3′ | 5′ GCAGAATTCC | 127 |
| SpCas9n | | GACATGACTC 3′ | |
| TargetAID_ | 5′AGG3′ | 5′ GCAGAATTCC | 128 |
| SpCas9n | | GACATGACTC 3′ | |
| BE3_SpCas9n- | 5′TGAC3′ | 5′ ATGGATGCAG | 129 |
| VQR | | AATTCCGACA 3′ | |
| BE4-Gam_ | 5′TGAC3′ | 5′ ATGGATGCAG | 130 |
| SpCas9n-VQR | | AATTCCGACA 3′ | |
| TargetAID_ | 5′TGAC3′ | 5′ ATGGATGCAG | 131 |
| SpCas9n-VQR | | AATTCCGACA 3′ | |
| BE3_ | 5′CAGGAT3′ | 5′ TGCAGAATTC | 132 |
| SaCas9nKKH | | CGACATGACT 3′ | |
| Target-AID_ | 5′CAGGAT3′ | 5′ TGCAGAATTC | 133 |
| SaCas9nKKH | | CGACATGACT 3′ | |

TABLE 5
Percentage of deamination with various Cas9 deaminase
for the cytidines present in the protospacer sequence

| Enzyme | C1 | C2 | C3 | C4 | C5 | C6 | C1 + C2 | C1 + C2 + C3 |
|---|---|---|---|---|---|---|---|---|
| BE3_SpCas9n | 0.05 | 1.97 | 0.04 | 0.02 | 0.03 | 0.01 | 0.01 | 0.02 |
| BE4-Gam_SpCas9n | 0.04 | 5.21 | 0.05 | 0.03 | 0.04 | 0.02 | 0.00 | 0.00 |
| Target AID_SpCas9n | 27.82 | 0.27 | 0.12 | 0.02 | 0.03 | 0.03 | 1.31 | 0.32 |
| BE3_SpCas9n-VQR | 0.12 | 0.63 | 0.05 | 0.04 | 0.02 | 0.03 | 0.08 | 0.00 |
| BE4-Gam_SpCas9n-VQR | 0.50 | 0.21 | 0.05 | 0.01 | 0.05 | 0.02 | 0.02 | 0.00 |
| Target AID_SpCas9n-VQR | 0.07 | 0.02 | 0.04 | 0.01 | 0.01 | 0.00 | 0.00 | 0.00 |
| BE3_SaCas9nKKH | 0.05 | 2.99 | 0.03 | 0.03 | 0.04 | 0.03 | 0.00 | 0.00 |
| Target-AID_SaCas9nKKH | 0.17 | 0.03 | 0.04 | 0.03 | 0.02 | 0.01 | 0.00 | 0.00 |
| Control GFP 1 | 0.05 | 0.03 | 0.06 | 0.02 | 0.03 | 0.06 | 0.00 | 0.00 |
| Control GFP 2 | 0.02 | 0.02 | 0.03 | 0.04 | 0.06 | 0.01 | 0.00 | 0.00 |
| 2nd exp. BE3_SpCas9n | 0.03 | 0.03 | 0.02 | 0.04 | 0.05 | 0.06 | 0.00 | 0.00 |
| 2nd exp. BE4-Gam_SpCas9n | 0.06 | 0.04 | 0.06 | 0.04 | 0.03 | 0.02 | 0.00 | 0.00 |
| 2nd exp. Target AID_SpCas9n | 18.48 | 0.19 | 0.09 | 0.03 | 0.03 | 0.02 | 1.69 | 0.11 |

### Example 5: Inducing the APP H684R Mutation

The H684R mutation required the deamination of the A in the CAT histidine codon in position 684 (FIG. 18) to produce the CGT codon for arginine. This was done using the adenine base editor (ABE)²³. This base editing enzyme used the SpCas9 enzyme and thus required a NGG PAM. There was such a PAM at 12 nucleotides from the target A (FIG. 18). Thus the target A was at position 9 in the protospacer sequence 5′TGAAGTTCATCATCAAAAAT3′ (SEQ ID NO: 134) targeted by a guide RNA (underlined in FIG. 18).

Since the efficacy of various ABEs varied depending of the position of the target A in the protospacer, we tested different ABE variants produced by Gaudelli et al.²³ (ABE6.3, ABE7.8, ABE7.9 and ABE7.10). Plasmids coding for these different adenosine base editors were obtained from AddGene Inc. respectively plasmids #102916, 102917, 102918 and 102674.

We also tested an adenosine deaminase fused with the KKH variant of SaCas9⁷¹ since there was an NNNRRT PAM (FIG. 18) at 19 nucleotides from the target A. The protospacer sequence targeted by the guide RNA is underlined and it is: CATCATCAAAAATTGGTACG (SEQ ID NO: 135).

The experiment was done in SH-SY5Y and exon 16 was PCR amplified and deep sequenced to establish the percentage of A to G mutation.

### Example 6: Inducing the APP H684R Mutation in the mRNA

To produce the desired H684R mutation, the A nucleotide in the CAT histidine codon in position 684 of the mRNA had to be modified into a G to produce the CGT codon for arginine. 100 000 SH-Sy5y neuroblastoma cells were plated, per well in a 24 wells plate. The following day, these cells were transfected with 400 ng of dPspCas13b-ADAR2DD (REPAIRv2, Addgene #103871) plasmid and 400 ng of Cas13 crRNA plasmid coding for a 50 nucleotides guide targeting the APP mRNA. Two versions of the guide RNA were tested targeting different parts of the APP mRNA. For one of the guide RNA, the target A was in front of position 35. For the other guide RNA the target A was in position 45. In the guide RNA, the nucleotide in front of the target A was a C (in bold in the sequences) to create a mismatch.

The sequence coding for a guide RNA in
which the mismatch is in position 45 is:
(SEQ ID NO: 136)
5′ GATGACGAACTTCATATCCTGAGTC
ATGTCGGAATTCTGCATCCATCTTC 3′
While the sequence coding for the guide
RNA in which the mismatch is in
position 35 is:
(SEQ ID NO: 137)
5′ GACCAATTTTTGATGACGAACTTCAT
ATCCTGAGTCATGTCGGAATTCTGC 3′

One control was transfected with 400 ng of dPspCas13b-ADAR2_{DD} (REPAIRv2) plasmid and 400 ng of eGFP plasmid instead of Cas13 crRNA plasmid. One other control was transfected with 800 ng of eGFP plasmid alone. 72 hrs after transfection, the RNA was extracted with Trizol reagent. For each sample, 1 μg of RNA was reverse transcribed. The cDNA of each sample was PCR amplified using primer specific for exon 14 and exon 17 of the APP cDNA. At the 5′ end of forward and at the 3′ end of the reverse primers, sequences required for Illumina deep sequencing were added.

The deep sequencing results indicated that the A nucleotide in the CAT histidine codon in the mRNA was modified into a G to produce the CGT codon for arginine in 1.3% of the sequences (Table 6). Thus it is possible to introduce by RNA base editing the H684R mutation, which is protective against AD.

TABLE 6
Percentage of H684R mutations

| | | His codon | Arg codon | |
|---|---|---|---|---|
| Sample | n | CAT | CGT | % |
| ADAR Guide35 | 99846 | 96939 | 1246 | 1.3 |
| ADAR Guide45 | 94104 | 91975 | 657 | 0.7 |
| Control Cas13 only | 89198 | 87059 | 131 | 0.2 |
| Control GFP | 93372 | 91770 | 84 | 0.1 |

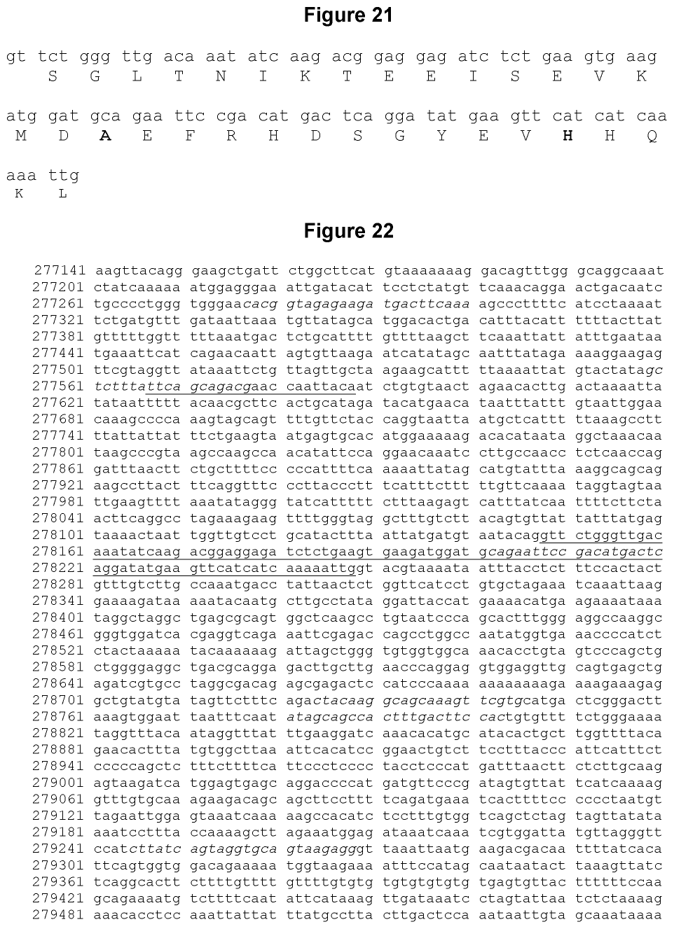

TABLE 7
Sequences described herein

| SEQ ID | |
|---|---|
| NO(s) | Description |
| 1, 2 | Human APP nucleotide and amino acid sequences |
| 3, 4 | BE3_SpCas9nVQR nucleotide and amino acid sequences |
| 5, 6 | BE3_SpCas9nEQR nucleotide and amino acid sequences |
| 7, 8 | BE3_SaCas9nKKH nucleotide and amino acid sequences |
| 9, 10 | YE1-BE3 nucleotide and amino acid sequences |
| 11, 12 | YE1-BE3_SpCas9nVQR nucleotide and amino acid |
| | sequences |
| 13, 14 | YE1-BE3_SpCas9nEQR nucleotide and amino acid |
| | sequences |
| 15, 16 | YE1-BE3_SaCas9nKKH |
| 17, 18 | BE4-GAM_SpCas9nVQR |
| 19, 20 | BE4-GAM_SpCas9nEQR |
| 21, 22 | Target-AID SpCas9nVQR nucleotide and amino acid |
| | sequences |
| 23, 24 | Target-AID SpCas9nEQR nucleotide and amino acid |
| | sequences |
| 25, 26 | Target-AID SaCas9nKKH nucleotide and amino acid |
| | sequences |
| 27, 28 | hADAR2 nucleotide and amino acid sequences |
| 29, 30 | REPAIRv2 nucleotide and amino acid sequences |
| 31, 32 | ABE6.3 nucleotide and amino acid sequences |
| 33, 34 | ABE7.8 nucleotide and amino acid sequences |
| 35, 36 | ABE7.9 nucleotide and amino acid sequences |
| 37, 38 | ABE7.10 nucleotide and amino acid sequences |
| 39, 40 | FIG. 18, sense and antisense sequences |
| 41 | FIG. 18, amino acid sequence |
| 42, 43 | FIG. 21, nucleotide and amino acid sequences |
| 44 | FIG. 22, nucleotide sequence |
| 45 | FIG. 2, amino acid sequence |
| 46, 47 | FIG. 5B, sense and antisense sequences |
| 48 | FIG. 5B, amino acid sequence |
| 49, 50 | FIG. 5C, sense and antisense sequences |
| 51 | FIG. 5C, amino acid sequence |
| 52 | FIG. 5A, nucleotide sequence |
| 53-76 | Target sequences for A673T mutation |
| 77-82 | Target sequences for A673V mutation |
| 83-94 | Target sequences for H684R mutation |
| 95-100 | Nuclear localization signals (NLSs) |
| 101-106 | Protein transduction domains (PTDs) |
| 107-117 | Endosomal escape peptides (Table 1) |
| 118-119 | TAT PDTs |
| 120 | Pep-1 PTD |
| 121, 122 | BCS1 and BCS2 primers |
| 123-125 | Target sequences |
| 126-133 | Protospacer sequences (Table 4) |
| 134, 135 | Protospacer sequences |
| 136, 137 | Guide RNA coding sequences |
| 138-148 | Guide RNA sequences |

The scope of the claims should not be limited by the preferred embodiments set forth in the examples, but should be given the broadest interpretation consistent with the description as a whole.

### REFERENCES

1. Ferri, CP, Prince, M, Brayne, C, Brodaty, H, Fratiglioni, L, Ganguli, M, et al. (2005). Global prevalence of dementia: a Delphi consensus study. Lancet 366: 2112-2117.
2. Plassman, BL, Langa, KM, Fisher, GG, Heeringa, SG, Weir, DR, Ofstedal, M B, et al. (2007). Prevalence of dementia in the United States: the aging, demographics, and memory study. Neuroepidemiology 29: 125-132.
3. Qiu, C, Kivipelto, M, and von Strauss, E (2009). Epidemiology of Alzheimer's disease: occurrence, determinants, and strategies toward intervention. Dialogues in clinical neuroscience 11: 111-128.
4. Masters, CL, Multhaup, G, Simms, G, Pottgiesser, J, Martins, R N, and Beyreuther, K (1985). Neuronal origin of a cerebral amyloid: neurofibrillary tangles of Alzheimer's disease contain the same protein as the amyloid of plaque cores and blood vessels. EMBO J 4: 2757-2763.
5. Zhang, YW, Thompson, R, Zhang, H, and Xu, H (2011). APP processing in Alzheimer's disease. Molecular brain 4: 3.
6. Hussain, I, Powell, D, Howlett, DR, Tew, DG, Meek, TD, Chapman, C, et al. (1999). Identification of a novel aspartic protease (Asp 2) as beta-secretase. Mol Cell Neurosci 14: 419-427.
7. Sinha, S, Anderson, JP, Barbour, R, Basi, GS, Caccavello, R, Davis, D, et al. (1999). Purification and cloning of amyloid precursor protein beta-secretase from human brain. Nature 402: 537-540.
8. Schmitt, HP (2006). Protein ubiquitination, degradation and the proteasome in neuro-degenerative disorders: no clear evidence for a significant pathogenetic role of proteasome failure in Alzheimer disease and related disorders. Med Hypotheses 67: 311-317.
9. Bignante, EA, Heredia, F, Morfini, G, and Lorenzo, A (2013). Amyloid beta precursor protein as a molecular target for amyloid beta-induced neuronal degeneration in Alzheimer's disease. Neurobiology of aging 34: 2525-2537.
10. Komor, AC, Zhao, KT, Packer, M S, Gaudelli, N M, Waterbury, A L, Koblan, L W, et al. (2017). Improved base excision repair inhibition and bacteriophage Mu Gam protein yields C:G-to-T:A base editors with higher efficiency and product purity. Sci Adv 3: eaao4774.
11. Kero, M, Paetau, A, Polvikoski, T, Tanskanen, M, Sulkava, R, Jansson, L, et al. (2013). Amyloid precursor protein (APP) A673T mutation in the elderly Finnish population. Neurobiology of aging 34: 1518 e1511-1513.
12. Jonsson, T, Atwal, JK, Steinberg, S, Snaedal, J, Jonsson, PV, Bjornsson, S, et al. (2012). A mutation in APP protects against Alzheimer's disease and age-related cognitive decline. Nature 488: 96-99.
13. Di Fede, G, Catania, M, Morbin, M, Giaccone, G, Moro, ML, Ghidoni, R, et al. (2012). Good gene, bad gene: new APP variant may be both. Prog Neurobiol 99: 281-292.
14. Cantu, L, Colombo, L, Stoilova, T, Deme, B, Inouye, H, Booth, R, et al. (2017). The A2V mutation as a new tool for hindering Abeta aggregation: A neutron and x-ray diffraction study. Scientific reports 7: 5510.
15. Kimura, A, Hata, S, and Suzuki, T (2016). Alternative Selection of beta-Site APP-Cleaving Enzyme 1 (BACE1) Cleavage Sites in Amyloid beta-Protein Precursor (APP) Harboring Protective and Pathogenic Mutations within the Abeta Sequence. J Biol Chem 291: 24041-24053.
16. Cox, DBT, Gootenberg, J S, Abudayyeh, O O, Franklin, B, Kellner, MJ, Joung, J, et al. (2017). RNA editing with CRISPR-Cas13. Science 358: 1019-1027.
17. Wettengel, J, Reautschnig, P, Geisler, S, Kahle, P J, and Stafforst, T (2017). Harnessing human ADAR2 for RNA repair—Recoding a PINK1 mutation rescues mitophagy. Nucleic Acids Res 45: 2797-2808.
18. St George-Hyslop, PH (2000). Molecular genetics of Alzheimer's disease. Biological psychiatry 47: 183-199.
19. Di Fede, G, Catania, M, Morbin, M, Rossi, G, Suardi, S, Mazzoleni, G, et al. (2009). A recessive mutation in the APP gene with dominant-negative effect on amyloidogenesis. Science 323: 1473-1477.
20. Giaccone, G, Morbin, M, Moda, F, Botta, M, Mazzoleni, G, Uggetti, A, et al. (2010). Neuropathology of the recessive A673V APP mutation: Alzheimer disease with distinctive features. Acta Neuropathol 120: 803-812.
21. Maloney, JA, Bainbridge, T, Gustafson, A, Zhang, S, Kyauk, R, Steiner, P, et al. (2014). Molecular mechanisms of Alzheimer disease protection by the A673T allele of amyloid precursor protein. J Biol Chem 289: 30990-31000.
22. Cimini, S, Sclip, A, Mancini, S, Colombo, L, Messa, M, Cagnotto, A, et al. (2016). The cell-permeable Abeta1-6A2VTAT(D) peptide reverts synaptopathy induced by Abeta1-42 wt. Neurobiol Dis 89: 101-111.
23. Gaudelli, N M, Komor, AC, Rees, HA, Packer, M S, Badran, AH, Bryson, D I, et al. (2017). Programmable base editing of A*T to G*C in genomic DNA without DNA cleavage. Nature 551: 464-471.
24. Anzalone, AV, Randolph, PB, Davis, JR, Sousa, AA, Koblan, LW, Levy, J M, et al. (2019). Search-and-replace genome editing without double-strand breaks or donor DNA. Nature.
25. Gaj, T, Gersbach, C A, and Barbas, CF, 3rd (2013). ZFN, TALEN, and CRISPR/Cas-based methods for genome engineering. Trends Biotechnol 31: 397-405.
26. Arnould, S, Delenda, C, Grizot, S, Desseaux, C, Paques, F, Silva, G H, et al. (2011). The I-Crel meganuclease and its engineered derivatives: applications from cell modification to gene therapy. Protein engineering, design & selection: PEDS 24: 27-31.
27. Menke, DB (2013). Engineering subtle targeted mutations into the mouse genome. Genesis 51: 605-618.
28. Mali, P, Yang, L, Esvelt, KM, Aach, J, Guell, M, DiCarlo, J E, et al. (2013). RNA-guided human genome engineering via Cas9. Science 339: 823-826.
29. Jinek, M, Chylinski, K, Fonfara, I, Hauer, M, Doudna, J A, and Charpentier, E (2012). A programmable dual-RNA-guided DNA endonuclease in adaptive bacterial immunity. Science 337: 816-821.
30. Cong, L, Ran, FA, Cox, D, Lin, S, Barretto, R, Habib, N, et al. (2013). Multiplex genome engineering using CRISPR/Cas systems. Science 339: 819-823.
31. Komor, AC, Kim, YB, Packer, M S, Zuris, J A, and Liu, DR (2016). Programmable editing of a target base in genomic DNA without double-stranded DNA cleavage. Nature 533: 420-424.
32. Hou, Z, Zhang, Y, Propson, N E, Howden, SE, Chu, LF, Sontheimer, E J, et al. (2013). Efficient genome engineering in human pluripotent stem cells using Cas9 from Neisseria meningitidis. Proc Natl Acad Sci USA 110: 15644-15649.
33. Deltcheva, E, Chylinski, K, Sharma, CM, Gonzales, K, Chao, Y, Pirzada, Z A, et al. (2011). CRISPR RNA maturation by trans-encoded small RNA and host factor RNase III. Nature 471: 602-607.
34. Sapranauskas, R, Gasiunas, G, Fremaux, C, Barrangou, R, Horvath, P, and Siksnys, V (2011). The Streptococcus thermophilus CRISPR/Cas system provides immunity in Escherichia coli. Nucleic Acids Res 39: 9275-9282.
35. Fonfara, I, Le Rhun, A, Chylinski, K, Makarova, K S, Lecrivain, A L, Bzdrenga, J, et al. (2014). Phylogeny of Cas9 determines functional exchangeability of dual-RNA and Cas9 among orthologous type II CRISPR-Cas systems. Nucleic Acids Res 42: 2577-2590.
36. Ran, FA, Cong, L, Yan, WX, Scott, DA, Gootenberg, J S, Kriz, A J, et al. (2015). In vivo genome editing using Staphylococcus aureus Cas9. Nature 520: 186-191.
37. P, C, N, J, and J M, J (2018). Minimal PAM specificity of a highly similar SpCas9 ortholog. Sci Adv 4.
38. Smith, T F, and Waterman, M S (1981). Comparison of biosequences. Adv Appl Math 2: 482-489.
39. Needleman, S B, and Wunsch, CD (1970). A general method applicable to the search for similarities in the amino acid sequence of two proteins. J Mol Biol 48: 443-453.
40. Pearson, W R, and Lipman, DJ (1988). Improved tools for biological sequence comparison. Proc Natl Acad Sci USA 85: 2444-2448.
41. Altschul, SF, Gish, W, Miller, W, Myers, E W, and Lipman, DJ (1990). Basic local alignment search tool. J Mol Biol 215: 403-410.
42. Mullan, M, Crawford, F, Axelman, K, Houlden, H, Lilius, L, Winblad, B, et al. (1992). A pathogenic mutation for probable Alzheimer's disease in the APP gene at the N-terminus of beta-amyloid. Nat Genet 1: 345-347.
43. Moro, ML, Giaccone, G, Lombardi, R, Indaco, A, Uggetti, A, Morbin, M, et al. (2012). APP mutations in the Abeta coding region are associated with abundant cerebral deposition of Abeta38. Acta Neuropathol 124: 809-821.
44. Janssen, JC, Beck, JA, Campbell, TA, Dickinson, A, Fox, N C, Harvey, R J, et al. (2003). Early onset familial Alzheimer's disease: Mutation frequency in 31 families. Neurology 60: 235-239.
45. Wakutani, Y, Watanabe, K, Adachi, Y, Wada-lsoe, K, Urakami, K, Ninomiya, H, et al. (2004). Novel amyloid precursor protein gene missense mutation (D678N) in probable familial Alzheimer's disease. J Neurol Neurosurg Psychiatry 75: 1039-1042.
46. Brouwers, N, Sleegers, K, and Van Broeckhoven, C (2008). Molecular genetics of Alzheimer's disease: an update. Ann Med 40: 562-583.
47. Hendriks, L, van Duijn, CM, Cras, P, Cruts, M, Van Hul, W, van Harskamp, F, et al. (1992). Presenile dementia and cerebral haemorrhage linked to a mutation at codon 692 of the beta-amyloid precursor protein gene. Nat Genet 1: 218-221.
48. Tomiyama, T, Nagata, T, Shimada, H, Teraoka, R, Fukushima, A, Kanemitsu, H, et al. (2008). A new amyloid beta variant favoring oligomerization in Alzheimer's-type dementia. Ann Neurol 63: 377-387.
49. Kamino, K, Orr, HT, Payami, H, Wijsman, E M, Alonso, M E, Pulst, S M, et al. (1992). Linkage and mutational analysis of familial Alzheimer disease kindreds for the APP gene region. Am J Hum Genet 51: 998-1014.
50. Grabowski, TJ, Cho, HS, Vonsattel, JP, Rebeck, G W, and Greenberg, SM (2001). Novel amyloid precursor protein mutation in an Iowa family with dementia and severe cerebral amyloid angiopathy. Ann Neurol 49: 697-705.
51. Carter, DA, Desmarais, E, Bellis, M, Campion, D, Clerget-Darpoux, F, Brice, A, et al. (1992). More missense in amyloid gene. Nat Genet 2: 255-256.
52. Pasalar, P, Najmabadi, H, Noorian, A R, Moghimi, B, Jannati, A, Soltanzadeh, A, et al. (2002). An Iranian family with Alzheimer's disease caused by a novel APP mutation (Thr714A1a). Neurology 58: 1574-1575.
53. Kumar-Singh, S, De Jonghe, C, Cruts, M, Kleinert, R, Wang, R, Mercken, M, et al. (2000). Nonfibrillar diffuse amyloid deposition due to a gamma(42)-secretase site mutation points to an essential role for N-truncated A beta(42) in Alzheimer's disease. Hum Mol Genet 9: 2589-2598.
54. Cruts, M, Dermaut, B, Rademakers, R, Van den Broeck, M, Stogbauer, F, and Van Broeckhoven, C (2003). Novel APP mutation V715A associated with presenile Alzheimer's disease in a German family. J Neurol 250: 1374-1375.
55. Ancolio, K, Dumanchin, C, Barelli, H, Warter, J M, Brice, A, Campion, D, et al. (1999). Unusual phenotypic alteration of beta amyloid precursor protein (betaAPP) maturation by a new Val-715->Met betaAPP-770 mutation responsible for probable early-onset Alzheimer's disease. Proc Natl Acad Sci USA 96: 4119-4124.
56. Guardia-Laguarta, C, Pera, M, Clarimon, J, Molinuevo, J L, Sanchez-Valle, R, Llado, A, et al. (2010). Clinical, neuropathologic, and biochemical profile of the amyloid precursor protein 1716F mutation. J Neuropathol Exp Neurol 69: 53-59.
57. Blauwendraat, C, Wilke, C, Jansen, IE, Schulte, C, Simon-Sanchez, J, Metzger, F G, et al. (2016). Pilot whole-exome sequencing of a German early-onset Alzheimer's disease cohort reveals a substantial frequency of PSEN2 variants. Neurobiology of aging 37: 208 e211-208 e217.
58. Terreni, L, Fogliarino, S, Franceschi, M, and Forloni, G (2002). Novel pathogenic mutation in an Italian patient with familial Alzheimer's disease detected in APP gene. Neurobiol Aging 23(1S): 319.
59. Eckman, CB, Mehta, N D, Crook, R, Perez-tur, J, Prihar, G, Pfeiffer, E, et al. (1997). A new pathogenic mutation in the APP gene (1716V) increases the relative proportion of A beta 42(43). Hum Mol Genet 6: 2087-2089.
60. Murrell, J, Farlow, M, Ghetti, B, and Benson, M D (1991). A mutation in the amyloid precursor protein associated with hereditary Alzheimer's disease. Science 254: 97-99.
61. Chartier-Harlin, MC, Crawford, F, Houlden, H, Warren, A, Hughes, D, Fidani, L, et al. (1991). Early-onset Alzheimer's disease caused by mutations at codon 717 of the beta-amyloid precursor protein gene. Nature 353: 844-846.
62. Goate, A, Chartier-Harlin, MC, Mullan, M, Brown, J, Crawford, F, Fidani, L, et al. (1991). Segregation of a missense mutation in the amyloid precursor protein gene with familial Alzheimer's disease. Nature 349: 704-706.
63. Murrell, JR, Hake, A M, Quaid, K A, Farlow, M R, and Ghetti, B (2000). Early-onset Alzheimer disease caused by a new mutation (V717L) in the amyloid precursor protein gene. Arch Neurol 57: 885-887.
64. Ghidoni, R, Albertini, V, Squitti, R, Paterlini, A, Bruno, A, Bernardini, S, et al. (2009). Novel T719P AbetaPP mutation unbalances the relative proportion of amyloid-beta peptides. J Alzheimers Dis 18: 295-303.
65. Wang, Q, Jia, J, Qin, W, Wu, L, Li, D, Wang, Q, et al. (2015). A Novel AbetaPP M722K Mutation Affects Amyloid-beta Secretion and Tau Phosphorylation and May Cause Early-Onset Familial Alzheimer's Disease in Chinese Individuals. J Alzheimers Dis 47: 157-165.
66. Kwok, JB, Li, QX, Hallupp, M, Whyte, S, Ames, D, Beyreuther, K, et al. (2000). Novel Leu723Pro amyloid precursor protein mutation increases amyloid beta42(43) peptide levels and induces apoptosis. Ann Neurol 47: 249-253.
67. Theuns, J, Marjaux, E, Vandenbulcke, M, Van Laere, K, Kumar-Singh, S, Bormans, G, et al. (2006). Alzheimer dementia caused by a novel mutation located in the APP C-terminal intracytosolic fragment. Hum Mutat 27: 888-896.
68. Kleinstiver, BP, Prew, M S, Tsai, SQ, Nguyen, NT, Topkar, VV, Zheng, Z, et al. (2015). Broadening the targeting range of Staphylococcus aureus CRISPR-Cas9 by modifying PAM recognition. Nat Biotechnol 33: 1293-1298.
69. Kleinstiver, BP, Prew, M S, Tsai, SQ, Topkar, W, Nguyen, NT, Zheng, Z, et al. (2015). Engineered CRISPR-Cas9 nucleases with altered PAM specificities. Nature 523: 481-485.
70. Nishida, K, Arazoe, T, Yachie, N, Banno, S, Kakimoto, M, Tabata, M, et al. (2016). Targeted nucleotide editing using hybrid prokaryotic and vertebrate adaptive immune systems. Science 353.
71. Yang, L, Zhang, X, Wang, L, Yin, S, Zhu, B, Xie, L, et al. (2018). Increasing targeting scope of adenosine base editors in mouse and rat embryos through fusion of TadA deaminase with Cas9 variants. Protein & cell.
72. Kim, YB, Komor, AC, Levy, J M, Packer, M S, Zhao, K T, and Liu, DR (2017). Increasing the genome-targeting scope and precision of base editing with engineered Cas9-cytidine deaminase fusions. Nat Biotechnol 35: 371-376.
73. Saito, T, Matsuba, Y, Mihira, N, Takano, J, Nilsson, P, Itohara, S, et al. (2014). Single App knock-in mouse models of Alzheimer's disease. Nat Neurosci 17: 661-663.

1. A method of decreasing Amyloid Precursor Protein (APP) processing into amyloidogenic Aβ peptide by a cell or the aggregation of the Aβ peptide, comprising providing said cell with:
a. a1) at least one guide RNA comprising a seed region of at least 18 consecutive nucleotides of a target sequence in an endogenous APP-encoding polynucleotide sequence present in said cell, wherein the target sequence of the guide RNA guide sequence is contiguous to a protospacer adjacent motif (PAM) in the endogenous APP gene polynucleotide sequence and wherein said PAM is recognized by a ribonucleoprotein complex comprising a Cas9 nickase; or
a2) a vector comprising a nucleic acid sequence corresponding to the guide RNA for expressing the guide RNA; and
b. b1) a Cas nickase-deaminase fusion protein comprising (i) a Cas nickase polypeptide domain and (ii) a cytidine deaminase polypeptide domain or an adenosine deaminase polypeptide domain; or
b2) a vector comprising a nucleic acid sequence encoding the Cas nickase-deaminase fusion protein, for expression of the Cas nickase-deaminase fusion protein; and
wherein at least one modification is introduced into the target sequence of the APP-encoding polynucleotide, wherein the at least one modification comprises modification of a cytidine into a thymidine or an adenosine into an inosine, and wherein the at least one modification decreases the processing of APP into amyloidogenic Aβ peptide by the cell or the aggregation of the Aβ peptide; and wherein if the APP-encoding nucleotide is mRNA and the at least one modification comprises deamination of an adenosine into an inosine, the gRNA further comprises a cytidine mismatch with the adenosine to be deaminated to an inosine in the target sequence.

2-4. (canceled)

5. The method of claim 1, wherein the at least one modification is located in intron 15, exon 16, intron 16, exon 17 and/or intron 17 of the APP-encoding polynucleotide sequence.

6. The method of claim 1, wherein said at least one modification results in at least one amino acid substitution in the region defined by amino acid positions 656 to 688 in the APP protein, wherein said amino acid positions are with respect to the APP protein sequence as set forth in SEQ ID NO: 2.

7. The method of claim 6, wherein said at least one amino acid substitution corresponds to at least one substitution at amino acid position 670, 671, 673, 678, 682, 684, 692, 693, 694, 704, 711, 712, 713, 714, 715 and/or 684 of the APP protein.

8-11. (canceled)

12. The method of claim 5, wherein said at least one modification corrects a mutation associated with an increased risk of developing Alzheimer's disease present in said endogenous APP polynucleotide gene sequence present in said cell.

13. The method of claim 5, wherein said at least one modification results in a mutation which reduces risk of developing Alzheimer's disease by reducing the formation or the aggregation of Amyloidogenic peptides.

14-26. (canceled)

27. A guide RNA as defined in claim 1.

28. An isolated nucleic acid comprising a nucleotide sequence corresponding to the guide RNA of claim 27 for expressing the guide RNA.

29. (canceled)

30. A vector comprising the isolated nucleic acid of claim 28.

31-50. (canceled)

51. A method of decreasing Amyloid Precursor Protein (APP) processing into amyloidogenic Aβ peptide by a cell or the aggregation of the Aβ peptide, comprising providing said cell with:
a. a1) at least one pegRNA comprising a seed region of at least 18 consecutive nucleotides of a target sequence in an endogenous APP-encoding polynucleotide sequence present in said cell, wherein the target sequence of the pegRNA guide sequence is contiguous to a protospacer adjacent motif (PAM) in the endogenous APP gene polynucleotide sequence and wherein said PAM is recognized by a ribonucleoprotein complex comprising a Cas9 nickase; or
a2) a vector comprising a nucleic acid sequence corresponding to the pegRNA for expressing the pegRNA; and
b. b1) a Cas nickase-reverse transcriptase fusion protein comprising (i) a Cas nickase polypeptide domain and (ii) a reverse transcriptase polypeptide domain; or
b2) a vector comprising a nucleic acid sequence encoding the Cas nickase-reverse transcriptase fusion protein, for expression of the Cas nickase-reverse transcriptase fusion protein; and
wherein at least one modification is introduced into the target sequence of the APP-encoding polynucleotide, wherein the at least one modification decreases the processing of APP into amyloidogenic Aβ peptide by the cell or the aggregation of the Aβ peptide.

52-54. (canceled)

55. The method of claim 51, wherein the at least one modification is located in intron 15, exon 16, intron 16, exon 17 and/or intron 17 of the APP-encoding polynucleotide sequence.

56. The method of claim 51, wherein said at least one modification results in at least one amino acid substitution in the region defined by amino acid positions 656 to 688 in the APP protein, wherein said amino acid positions are with respect to the APP protein sequence as set forth in SEQ ID NO: 2.

57. The method of claim 56, wherein said at least one amino acid substitution corresponds to at least one substitution at amino acid position 670, 671, 673, 678, 682, 684, 692, 693, 694, 704, 711, 712, 713, 714, 715 and/or 684 of the APP protein.

58-61. (canceled)

62. The method of claim 55, wherein said at least one modification corrects a mutation associated with an increased risk of developing Alzheimer's disease present in said endogenous APP polynucleotide gene sequence present in said cell.

63. The method of claim 55, wherein said at least one modification results in a mutation which reduces risk of developing Alzheimer's disease by reducing the formation or the aggregation of Amyloidogenic peptides.

64-76. (canceled)

77. A guide RNA as defined in claim 51.

78. An isolated nucleic acid comprising a nucleotide sequence corresponding to the guide RNA of claim 77 for expressing the guide RNA.

79. (canceled)

80. A vector comprising the isolated nucleic acid of claim 78.

81-100. (canceled)

101. The method of claim 7, wherein said at least one amino acid substitution corresponds to at least one substitution at amino acid position 673 and/or 684 of the APP protein.

101. The method of claim 57, wherein said at least one amino acid substitution corresponds to at least one substitution at amino acid position 673 and/or 684 of the APP protein.
